(19) United States
(12) Patent Application Publication
(10) Pub. No.: US 2016/0174858 A1
(43) Pub. Date: Jun. 23, 2016

(54) NON-INVASIVE INTRACRANIAL PRESSURE MONITORING SYSTEM AND METHOD THEREOF

(71) Applicant: Vivonics, Inc., Sudbury, MA (US)

(72) Inventor: Anna M. Galea, Stow, MA (US)

(21) Appl. No.: 14/995,790

(22) Filed: Jan. 14, 2016

Related U.S. Application Data
(63) Continuation-in-part of application No. 13/939,824, filed on Jul. 11, 2013.

(51) Int. Cl. A61B 5/03; A61B 5/00; A61B 5/024

(52) U.S. Cl. CPC A61B 5/031; A61B 5/02416; A61B 5/02438; A61B 5/0059; A61B 5/7246; A61B 5/7278

(57) ABSTRACT

A non-invasive pressure monitoring system includes a first sensor placed proximate to a perfusion field of an artery receiving blood which emanates from the cranial cavity is configured to measure pulsations of the artery receiving blood which emanates from the cranial cavity artery and generate first output signals. A second sensor placed proximate to a perfusion field of an artery which does not receive blood emanating from the cranial cavity configured to measure pulsations of the artery which does not receive blood emanating from the cranial cavity and generate second output signals. A processing subsystem responsive to the first output signal and the second output signal is configured to calculate the time shift associated with the highest cross-correlation of the two signals, or the phase shift or magnitudes of different frequencies included in the first output signals and the second output signals and determine intracranial pressure of the human subject from a time shift of the cross-correlation with the highest value.

### RELATED APPLICATIONS

This application is a continuation-in-part application of U.S. patent application Ser. No. 13/939,824 filed Jul. 11, 2013, and claims the benefit of and priority thereto under 35 U.S.C. §§119, 120, 363, 365, and 37 C.F.R. §1.55 and §1.78, which is incorporated herein by this reference.

### GOVERNMENT RIGHTS

This invention was made with government support under W81XWH-13-C-00187 awarded by the U.S. Army, and M67854-15-C-6528 awarded by the U.S. Marine Corps. The government has certain rights in the invention.

### FIELD OF THE INVENTION

This invention relates to a non-invasive intracranial pressure monitoring system and method thereof.

### BACKGROUND OF THE INVENTION

A closed-head brain injury, whether incurred as a result of blunt force trauma or a blast wave, can have insidious effects on a person. Although many casualties may suffer from headache or dizziness, it is difficult with conventional systems and methods to image every soldier or athlete in the field who experiences a potential brain injury. Most conventional imaging methods are large and require significant power. Moreover, damage to delicate brain tissues is frequently undetectable by conventional imaging, including CT scanning, even when such imaging is available.

The brain, however, is a soft organ with delicate structures held within a fixed volume. Damage to the small structures within a brain cause local swelling and cerebral blood flow and systemic blood pressure may not necessarily decrease with brain swelling. Therefore, even mild swelling of about 1 to 3 cc of extra fluid results in increased pressure. This elevated intracranial pressure (ICP) can itself cause more damage, including brain cell death and permanent brain injury or death.

Intracranial pressure (ICP) is the pressure on brain and the cerebrospinal fluid (CSF) within the cranium. It is a fundamental physiological parameter with the same importance as arterial blood pressure. Increased ICP refers to a serious condition in which there is an increase in fluid pressure inside the skull, whether blood or cerebrospinal fluid. In children, causes of increased ICP, commonly known as intracranial hypertension (IH), include, traumatic brain injury, intracranial tumors or hemorrhage, hydrocephalus due to ventricular shunt failure, cerebral infraction, infections, and untreated craniosynostosis. Raised ICP complicates both traumatic and non-traumatic encephalopathies. It causes impaired cerebral perfusion leading to brain ischemia and may result in death due to global ischemia or herniation of brain tissue.¹ Timely recognition and management of RI may improve patient outcome. However, the standard tools for monitoring ICP are invasive, require a high level of expertise and have clinically significant risks.

In many active populations, especially true of the armed forces, or professional sports, a casualty may try to shrug off the seemingly mild symptoms of headache, dizziness, and the like. However, an unknown percentage of these injured are experiencing clinically significant elevated ICP which may worsen or result in permanent damage which could otherwise be avoided with the appropriate application of pharmacological or surgical interventions.

Currently, there is no known robust, portable, and reliable system or method which can accurately monitor ICP without direct access to the intracranial space. Therefore, it may not be feasible to check ICP on every person who has or may have experienced trauma to the brain. It is unknown how many casualties of blunt or blast trauma have underlying increased pressure in the brain that occurs in response to the injury.

The best conventional systems currently available to identify which casualties are at the most risk of brain injury are those that monitor the physical trauma (such as blast waves or impact) the head experiences. However, such conventional systems may only provide information based on an empirical diagnostic technique which may not take into account individual variability with regards to susceptibility of brain injury. Thus, two people experiencing the same physical trauma are likely to exhibit different levels of damage, but without a direct measure of the damage, they may be impossible to differentiate.

There are many conventional systems and methods that may hold promise for being able to measure or monitor ICP without direct access to the brain. These conventional systems and methods often employ large, heavy, power intensive equipment, such as MRI, and the like, and therefore are not portable. This limits their use in the battlefield or at the sidelines in sports related injuries.

Thus, there is a need for a system and method that can measure ICP noninvasively, unobtrusively and continuously to provide an accurate measure of the extent of brain injury and enable medical care to timely provide the needed care. Moreover, in cases where the injury might have gone undetected until extensive damage has been done due to unchecked swelling, there is a need for effective threat agent that more quickly resolves the problem and returns the injured person to work, a soldier to duty, or a athlete to top performance.

The supraorbital artery provides an avenue of information from the cranial cavity. This vessel emanates from the ophthalmic artery, which in turn comes from the internal carotid artery. It is accessible at the forehead after it exits from the orbit. By virtue of its path along the periphery of the brain, it carries with it information related to the ICP. A signal from this artery can be compared to a signal that is similar to it in order to calculate the intracranial pressure. For example a signal from the temporal artery, which emanates from the external carotid and is measured at the level of the head will have traveled the same distance from the heart and through much of the same vasculature, save for the last short part of travel which is internal to the cranium for the supraorbital signal and external to the cranium for the temporal artery.

U. S. Pub. No. 2009/0143656 to Manwaring et al., incorporated by reference herein, discloses that the supraorbital artery may be used to determine ICP. However, as disclosed therein, the '656 patent application teaches a single phase shift is sought between a signal obtained at the supraorbital artery and one from another source. To date, no practical device has emerged from the '656 patent application. Novel algorithms that determine intracranial pressure from the available signals which can be provided by sensors proximate the supraorbital artery or similar intracranial artery or the external carotid artery or one of its branches can yield a compact device that calculates intracranial pressure and meet the above stated unmet need.

### SUMMARY OF THE INVENTION

In one aspect, a non-invasive intracranial pressure monitoring system is featured including a first sensor placed proximate to a perfusion field of an artery of a human subject receiving blood which emanates from the cranial cavity configured to measure pulsations of the artery receiving blood which emanates from the cranial cavity artery and generate first output signals. A second sensor placed proximate to a perfusion field of an artery of a human subject which does not receive blood emanating from the cranial cavity is configured to measure pulsations of the artery which does not receive blood emanating from the cranial cavity and generate second output signals. A processing subsystem responsive to the first output signals and the second output signals is configured to calculate a cross-correlation of the first output signals and the second output signals, and determine the intracranial pressure of the human subject from a time shift of the cross-correlation with the highest value.

In one embodiment, the first sensor and the second sensor may include near infrared (NIR) sensors. The first sensor and the second sensor may include pressure sensors. The first sensor and the second sensor may include photoplethysmographic sensors. The first sensor and the second sensor may include a combination of one or more of a near infrared (NIR) sensor, a pressure sensor and a photoplethysmographic sensor. The first sensor may be placed proximate one of the supraorbital artery, the supratrocheal artery, or the ophthalmic artery. The second sensor may be placed proximate the external carotid artery or one of its branches.

In another aspect, a non-invasive intracranial pressure monitoring system is featured including a first sensor placed proximate to a perfusion field of an artery of a human subject receiving blood which emanates from the cranial cavity configured to measure pulsations of the artery receiving blood which emanates from the cranial cavity artery and generate first output signals. A second sensor placed proximate to a perfusion field of an artery of the human subject which does not receive blood emanating from the cranial cavity is configured to measure pulsations of the artery which does not receive blood emanating from the cranial cavity and generate second output signals. A processing subsystem responsive to the first output signal and the second output signal is configured to calculate the phase shift of different frequencies included in the first output signals and the second output signals, and determine intracranial pressure of the human subject from the phase shift at the different frequencies of the first output signals and the second output signals.

In another embodiment, the first sensor and the second sensor may include near-infrared (NM) sensors. The first sensor and the second sensor may include pressure sensors. The first sensor and the second sensor may include photoplethysmographic sensors. The first sensor and the second sensor may include a combination of one or more of a near infrared (NIR) sensor, a pressure sensor and a photoplethysmographic sensor. The first sensor may be placed proximate one of the supraorbital artery, the supratrocheal artery, or the ophthalmic artery. The second sensor may be placed proximate the external carotid artery or one of its branches.

In another aspect, a non-invasive intracranial pressure monitoring system is featured including a first sensor placed proximate to a perfusion field of an artery of a human subject receiving blood which emanates from the cranial cavity configured to measure pulsations of the artery receiving blood which emanates from the cranial cavity artery and generate first output signals. A second sensor placed proximate to a perfusion field of an artery of the human subject which does not receive blood emanating from the cranial cavity is configured to measure pulsations of the artery which does not receive blood emanating from the cranial cavity and generate second output signals. A processing subsystem responsive to the first output signal and the second output signal is configured to calculate a magnitude of different frequencies included in the first output signals and the second output signals, and determine intracranial pressure of the human subject from a difference in magnitude at the different frequencies of the first output signals and the second output signals.

In yet another embodiment, the first sensor and the second sensor may include near-infrared (NIR) sensors. The second sensor may include pressure sensors. The first sensor and the second sensor may include photoplethysmographic sensors. The first sensor and the second sensor may include a combination of one or more of a near infrared (NIR) sensor, a pressure sensor and a photoplethysmographic sensor. The first sensor may be placed proximate one of the supraorbital artery, the supratrocheal artery, or the ophthalmic artery. The second sensor may be placed proximate the external carotid artery or one of its branches.

In yet another aspect, a non-invasive intracranial pressure monitoring system is featured including a first sensor placed proximate to a perfusion field of an artery of a human subject receiving blood which emanates from the cranial cavity configured to measure pulsations of the artery receiving blood which emanates from the cranial cavity artery and generate first output signals. A second sensor placed proximate to a perfusion field of an artery of the human subject which does not receive blood emanating from the cranial cavity is configured to measure pulsations of the artery which does not receive blood emanating from the cranial cavity and generate second output signals. A processing subsystem responsive to the first output signals and the second output signals is configured to calculate a difference between the first output signals and second output signals, and determine the intracranial pressure from the difference.

In another embodiment, the first sensor and the second sensor may include near-infrared sensors. The first sensor and the second sensor may include pressure sensors. The first sensor and the second sensor may include photoplethysmographic sensors. The first sensor and the second sensor may include a combination of one or more of a near infrared (NIR) sensor, a pressure sensor and a photoplethysmographic sensor. The first sensor may be placed proximate one of the supraorbital artery, the supratrocheal artery, or the ophthalmic artery. The second sensor may be placed proximate the external carotid artery or one of its branches.

In another aspect, a method for non-invasively determining intracranial pressure is featured, the method including measuring pulsations of the artery of a human subject receiving blood which emanates from the cranial cavity artery and generating first output signals. The method includes measuring pulsations of the artery of the human subject which does not receive blood emanating from the cranial cavity and generate second output signals. The method includes calculating a cross-correlation of the first output signals and the second output signals determining the intracranial pressure of the human subject from a time shift of the cross-correlation with the highest value.

In another embodiment, the measuring pulsations of the artery of a human subject receiving blood which emanates from the cranial cavity artery may be performed proximate one of the supraorbital artery, the supratrocheal artery, or the ophthalmic artery. The measuring pulsations of the artery of the human subject which does not receive blood emanating from the cranial cavity may be performed proximate the external carotid artery or one of its branches.

In another aspect, a method for non-invasively determining intracranial pressure is featured, the method including measuring pulsations of the artery of a human subject receiving blood which emanates from the cranial cavity artery and generating first output signals. The method includes measuring pulsations of the artery of the human subject which does not receive blood emanating from the cranial cavity and generate second output signals. The method includes calculating the phase shift of different frequencies included in the first output signals and the second output signals and determining intracranial pressure of the human subject from the phase shift at the different frequencies of the first output signals and the second output signals.

In one embodiment, the measuring pulsations of the artery of a human subject receiving blood which emanates from the cranial cavity artery may be performed proximate one of the supraorbital artery, the supratrocheal artery, or the ophthalmic artery. The measuring pulsations of the artery of the human subject which does not receive blood emanating from the cranial cavity may be performed proximate the external carotid artery or one of its branches.

In another aspect, a method for non-invasively determining intracranial pressure is featured, the method includes measuring pulsations of the artery of a human subject receiving blood which emanates from the cranial cavity artery and generating first output signals. The method includes measuring pulsations of the artery of the human subject which does not receive blood emanating from the cranial cavity and generate second output signals. The method includes calculating a magnitude of different frequencies included in the first output signals and the second output signals and determining intracranial pressure of the human subject from a difference in magnitude at the different frequencies of the first output signals and the second output signals.

In one embodiment, measuring pulsations of the artery of a human subject receiving blood which emanates from the cranial cavity artery may be performed proximate one of the supraorbital artery, the supratrocheal artery, or the ophthalmic artery. The measuring pulsations of the artery of the human subject which does not receive blood emanating from the cranial cavity may be performed proximate the external carotid artery or one of its branches.

In another aspect, a method for non-invasively determining intracranial pressure is featured including measuring pulsations of the artery of a human subject receiving blood which emanates from the cranial cavity artery and generating first output signals. The method includes measuring pulsations of the artery of the human subject which does not receive blood emanating from the cranial cavity and generate second output signals. The method includes calculating a difference between the first output signals and second output signal and determining the intracranial pressure from the difference.

In one embodiment, the measuring pulsations of the artery of a human subject receiving blood which emanates from the cranial cavity artery may be performed proximate one of the supraorbital artery, the supratrocheal artery, or the ophthalmic artery. The measuring pulsations of the artery of the human subject which does not receive blood emanating from the cranial cavity may be performed proximate the external carotid artery or one of its branches.

### BRIEF DESCRIPTION OF THE SEVERAL VIEWS OF THE DRAWINGS

Other objects, features and advantages will occur to those skilled in the art from the following description of a preferred embodiment and the accompanying drawings, in which:

FIG. 1 shows a depiction of a vasculature of the human head;

FIG. 2 is a three-dimensional view showing the primary components of one embodiment of the non-invasive intracranial pressure monitoring system and method thereof of this invention;

FIG. 3 is a view showing exemplary locations for the placement of the first sensor shown in FIG. 2;

FIG. 4 is a view showing exemplary locations for the placement of the second sensor shown in FIG. 2;

FIGS. 5 and 6 are views showing exemplary locations for placement of the third sensor shown in FIG. 2;

FIG. 7 is a flowchart showing the primary steps of one embodiment of the method of non-invasively determining the intracranial pressure of this invention;

FIG. 8 is a photograph showing an enlarged view of one example of the processing subsystem and the third sensor shown in FIG. 2;

FIG. 9 is a schematic diagram showing an example of the first sensor, second sensor, and/or third sensor shown in FIG. 2;

FIG. 10 shows an example of a blood pressure cuff and blood pressure cuff subsystem which may be utilized by the third sensor shown in FIG. 2 to determine ICP;

FIG. 11 is flow chart showing the primary steps of one embodiment of the method for non-invasively determining the intracranial pressure of this invention;

FIG. 12 is flow chart showing in further detail the steps of method for non-invasively determining the intracranial pressure monitoring shown in FIG. 11;

FIG. 13 is flow chart showing the primary steps of another embodiment of the method for non-invasively determining the intracranial pressure of this invention;

FIG. 14 is flow chart showing the primary steps of yet another embodiment of the method for non-invasively determining the intracranial pressure of this invention;

FIG. 15 is a schematic block diagram overview showing the primary components used by the method for non-invasively determining the intracranial pressure shown in one or more of FIGS. 7 and 11-14;

FIG. 16 is a schematic block diagram overview showing the primary components used by the method for non-invasively determining the intracranial pressure shown in one or more of FIGS. 7 and 11-14;

FIG. 17 is a graph showing exemplary test results of the non-invasive intracranial pressure system and method thereof shown in one or more of FIGS. 2-16;

FIG. 18 shows graphs showing exemplary test results of the non-invasive intracranial pressure system and method thereof shown in one or more of FIGS. 2-16;

FIG. 19 is a graph showing exemplary test results of the non-invasive intracranial pressure system and method thereof shown in one or more of FIGS. 2-16;

FIG. 20 shows an example of the third sensor shown in FIG. 2 configured as an electrocardiogram sensor and also showing an electrocardiogram sensor subsystem;

FIG. 21 is a schematic diagram depicting one example of the system and method thereof shown in one or more of FIGS. 2-20 for measuring ICP based on a time lag between signals from the first sensor, the second sensor, and the third sensor;

FIG. 22 is a three-dimensional view of one example of a hand-held device configured to hold the first sensor and second sensor shown in at least

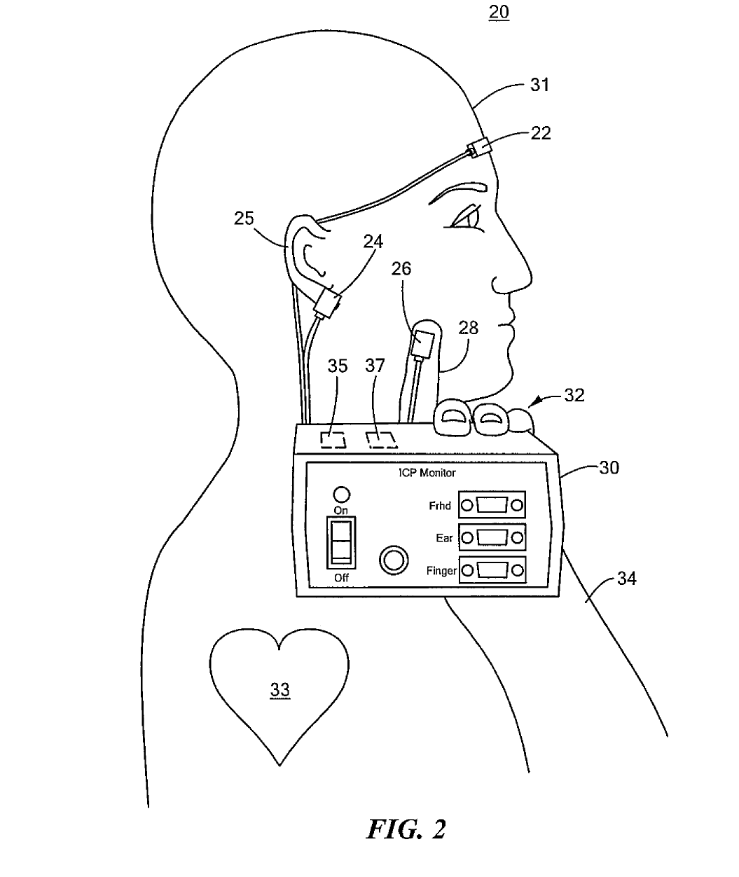

FIG. 2 in a spaced orientation;

FIG. 23 is a three-dimensional view of one example of the system and method thereof shown in one or more of FIGS. 2-21 integrated as a hand-held device;

FIG. 24 is a schematic block diagram showing one example of a feature extractor and artificial neural network which may be used to determine ICP in accordance with one embodiment of this invention;

FIG. 25 is a three-dimensional view showing the primary components of another embodiment of the non-invasive intracranial pressure monitoring system and method thereof of invention;

FIG. 26 shows an example of the first and/or second sensor shown in FIG. 25 configured as a photoplethysmographic sensor;

FIG. 27 shows exemplary first output signals and second output signals generated by the first and second sensors shown in FIG. 25;

FIG. 28 is a flowchart showing the primary steps of one embodiment for the method for non-invasively determining the intracranial pressure for the system shown in FIG. 25;

FIG. 29 shows additional exemplary first output signals and second output signals generated by the sensors shown in FIG. 25;

FIG. 30 is a flowchart showing the primary steps of another embodiment of the method for non-invasively determining intracranial pressure for the system shown in FIG. 25;

FIG. 31 is a flowchart showing the primary steps of another embodiment of the method for non-invasively determining intracranial pressure for the system shown in FIG. 25; and

FIG. 32 is a flowchart showing the primary steps of another embodiment of the method for non-invasively determining intracranial pressure for the system shown in FIG. 25.

### DETAILED DESCRIPTION OF THE INVENTION

Aside from the preferred embodiment or embodiments disclosed below, this invention is capable of other embodiments and of being practiced or being carried out in various ways. Thus, it is to be understood that the invention is not limited in its application to the details of construction and the arrangements of components set forth in the following description or illustrated in the drawings. If only one embodiment is described herein, the claims hereof are not to be limited to that embodiment. Moreover, the claims hereof are not to be read restrictively unless there is clear and convincing evidence manifesting a certain exclusion, restriction, or disclaimer.

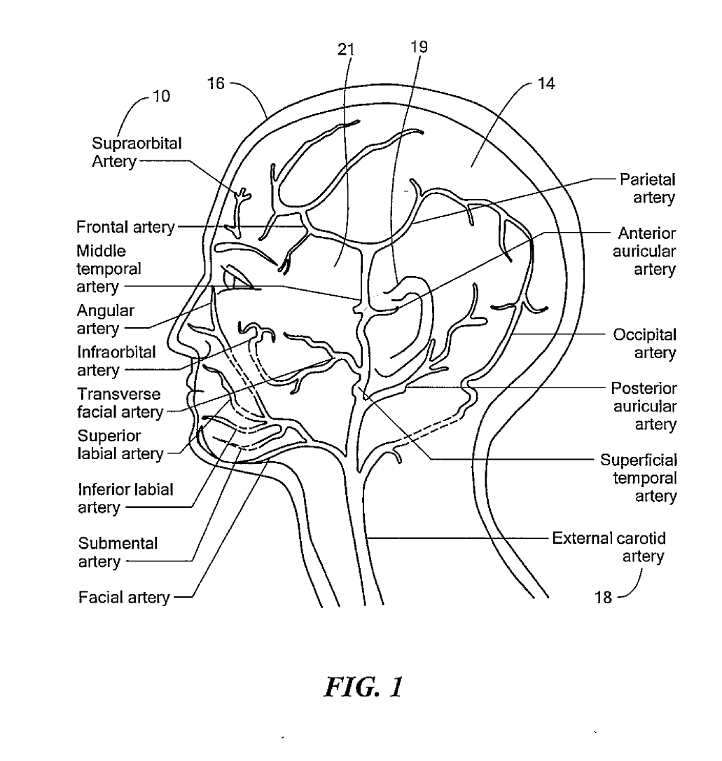

FIG. 1 shows an example of the vasculature of the human head. One key vasculature often used in determining ICP is supraorbital artery 10. Supraorbital artery 10 is an example of an artery which receives a flow of blood which emanates from within cranial cavity 14. As can be seen, supraorbital artery 10 is proximate forehead 16 of the skull. External carotid artery 18 or one of its branches as shown is another artery often used to determine ICP. As can be seen, external carotid artery 18 is branched and is an example of an artery which does not receive blood which emanates from cranial cavity 14. External carotid artery 18 is located proximate to ear 19 or temple 21.

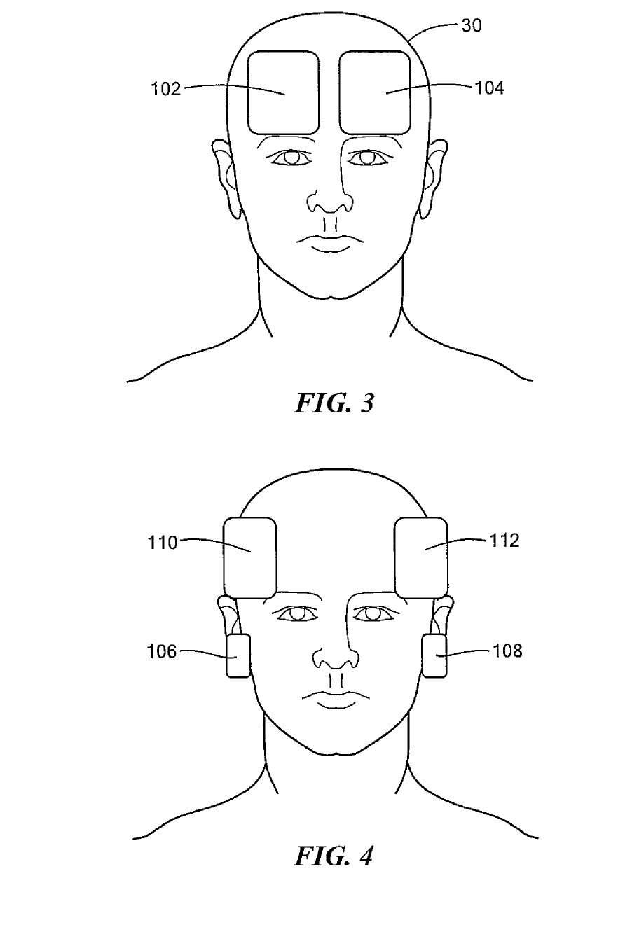

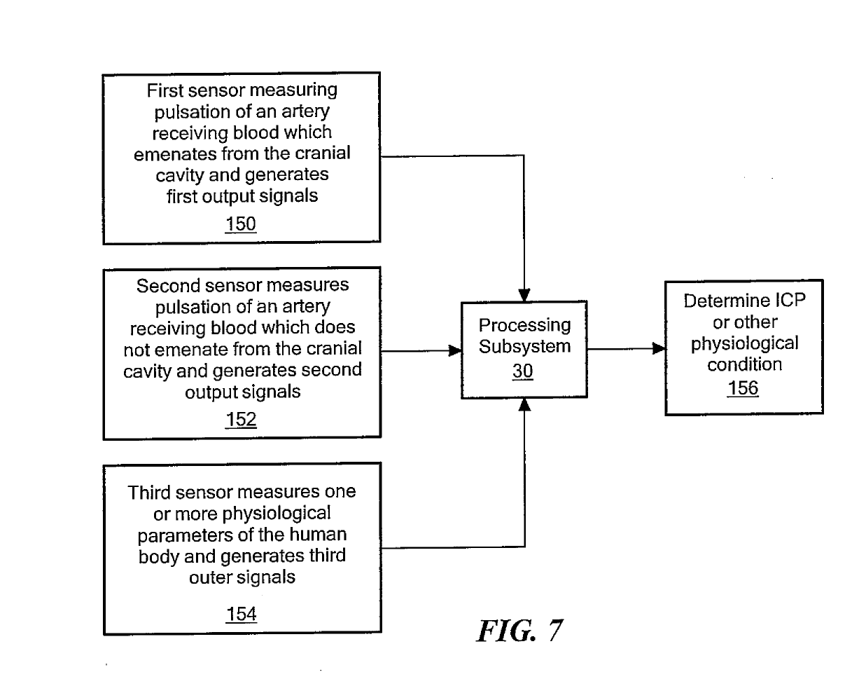

Non-invasive intracranial pressure monitoring system 20, FIG. 2, and the method thereof, FIG. 7, of one embodiment of this invention, includes first sensor 22 placed proximate a diffusion field of an artery receiving blood which emanates from within cranial cavity 14, FIG. 1, and is configured to measure pulsations of that artery and generate first output signals. In one example, the diffusion field is a capillary bed and the artery receiving blood which emanates from the cranial cavity is supraorbital artery 10. In this example, first sensor 22, FIG. 2, is placed proximate forehead 30, e.g., at one of locations 102 or 104, FIG. 3, which is near supraorbital artery 10, FIG. 1.

Non-invasive intracranial pressure monitoring system 20, FIG. 2, also includes second sensor 24, FIG. 2, placed proximate a perfusion field of an artery which does not receive blood emanating from cranial cavity 14 and is configured to measure pulsations of that artery and generate second output signals. In one example, second sensor 24 may be placed approximately the same distance from the heart 33 as first sensor 22. In one example, the diffusion field is a capillary bed and the artery receiving blood which does not emanate from the cranial cavity is external carotid artery 18 or one of its branches, FIG. 1. For example, sensor 24 may be placed proximate ear 25, FIG. 2, as shown, e.g., on the ear lobe, which is near external carotid artery 18 or at one of locations 106 or 108, FIG. 4. In other examples, second sensor 24 may be placed on or near the temple, e.g., at one of locations at one of 110 or 112, FIG. 4.

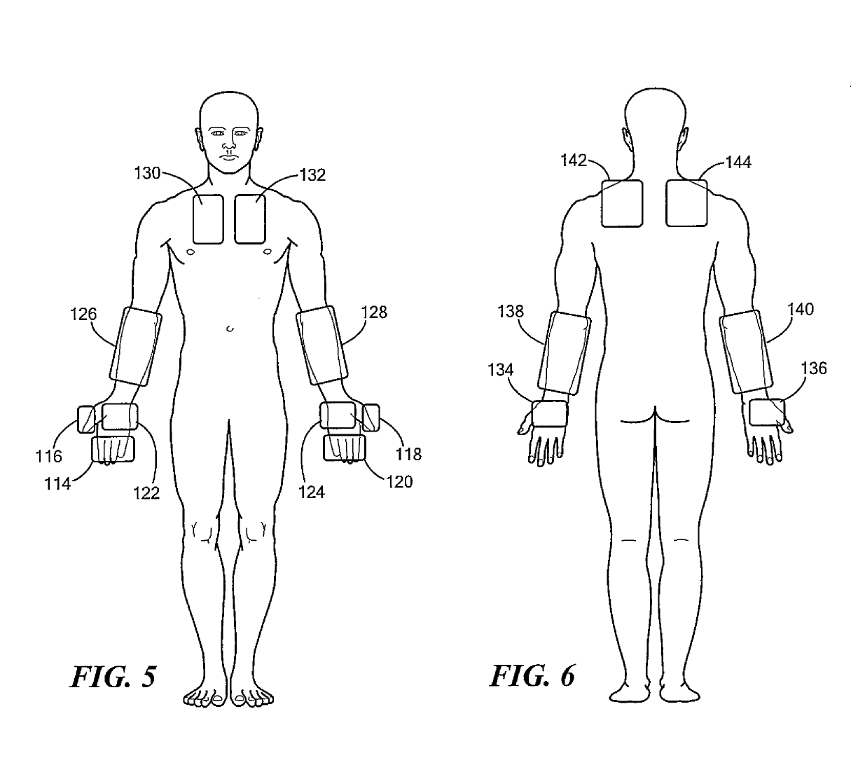

Non-invasive intracranial pressure monitoring system also includes third sensor 26, FIG. 2, configured to measure one or more physiological parameters of the human body and generate third output signals. In one example, the one or more physiological parameters may include pulsations of a distal artery. In this case, third sensor 26 may be placed on finger 28, as shown, or at one of finger locations 114, 116, 118 or 120, FIG. 5, which are located near one or more distal arteries inside the fingers. In other examples, third sensor 26 may be placed proximate the transverse cervical artery, the radial artery, or similar type distal artery, e.g., on the front or back of the hand, the front and back of the forearm, or the front and back of the torso, e.g., at one of locations 122, 124, 126, 128, 130 or 132, FIG. 5, or locations 134, 136, 138, 140 142, 144, FIG. 6, or 136, FIG. 6, or any other desired distal location of the human body.

Non-invasive intracranial pressure monitoring system 20 also includes processing subsystem 30, FIG. 2, responsive to the first output signals from first sensor 22, the second output signals from second sensor 24, and the third output signals from third sensor 26, respectively, to determine the intracranial pressure (ICP). Processing subsystem 30 includes one or more processors, e.g., processor 35 (shown in phantom). Processing subsystem 30 also includes one or more programs stored in a memory, e.g., memory 37 (shown in phantom), which are configured to be executed by the one or more processors. The one or more programs include instructions to determine ICP. The first output signals, second output signals, and the third output signals include data on the measured pulsations of the artery receiving blood which emanates from the cranial cavity, the artery receiving blood which does not emanate from the cranial cavity, and the distal artery, respectively, to determine the ICP.

FIG. 7 shows a flowchart of one embodiment of the method of determining ICP using non-invasive intracranial pressure monitoring system 20, FIG. 2. In this example, first sensor 22 measures pulsations of an artery receiving blood which emanates from the cranial cavity and generates the first output signals, step 150. Second sensor 24 measures pulsations of an artery which does not emanate from the cranial cavity and generates the second output signals, step 152. Third sensor 26 measures one or more physiological parameters of the human body and generates the third output signals, step 154. Processing subsystem 30 is responsive to the first output signals, the second output signals, and the third output signals and determines ICP or other physiological conditions, e.g., as a stroke, as discussed below, step 156.

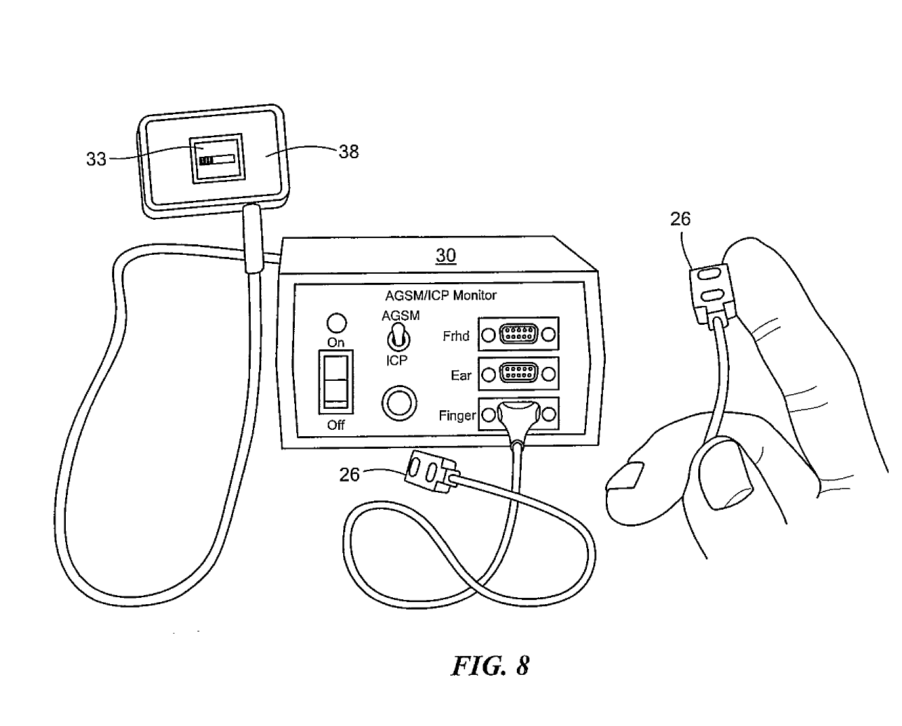

FIG. 8 shows an enlarged view of one example of processor subsystem 30 and enlarged view of one example of third sensor 26 when it is configured to measure pulsations of a distal artery as discussed above, coupled to processing subsystem 30. In one design, first sensor 22, FIG. 2, second sensor 24 and third sensor 26 are preferably configured as a near infrared (NIR) sensor and measure signals in the near infrared range, e.g., about 750 nm to about 950 nm. Sensor 26 shows an example of such an NIR sensor, which, in this example, was fabricated by Vivonics Inc., Sudbury Mass. System 20 also preferably includes monitor 38, FIG. 3, e.g., small LCD screen 33, configured to display and provide real-time feedback of the determined ICP values.

In another embodiment, first sensor 22, second sensor 24 and/or third sensor 26 may be configured as a pressure sensor, e.g., pressure sensor 148, FIG. 8, available from Tekscan Inc., South Boston, Mass., configured to measure pressure signals and generate the first output signals, the second output signals and/or the third output signals. In this example, to measure ICP, first sensor 22 may be preferably placed proximate the supraorbital artery 10, FIG. 1, e.g., at one of locations 102 or 104, FIG. 3, as discussed above, second sensor 24 may be placed proximate the external carotid artery or one of its branches, such as the temporal artery or facial artery, e.g., at one of locations 106, 108, 110, or 112, FIG. 4 and third sensor 26 may be placed on a distal artery, such as the transverse cervical artery, radial artery, or similar distal artery, e.g., at one of locations 114-144, FIGS. 5 and 6, discussed above. In this design, first sensor 22, second sensor 24, and third sensor 26 measure a signal proportional to the amount of blood in the proximate artery and generate the first, second and third output signals. The signal processing to determine ICP using processing subsystem 30 is similar to that recorded by NIR type sensors discussed above.

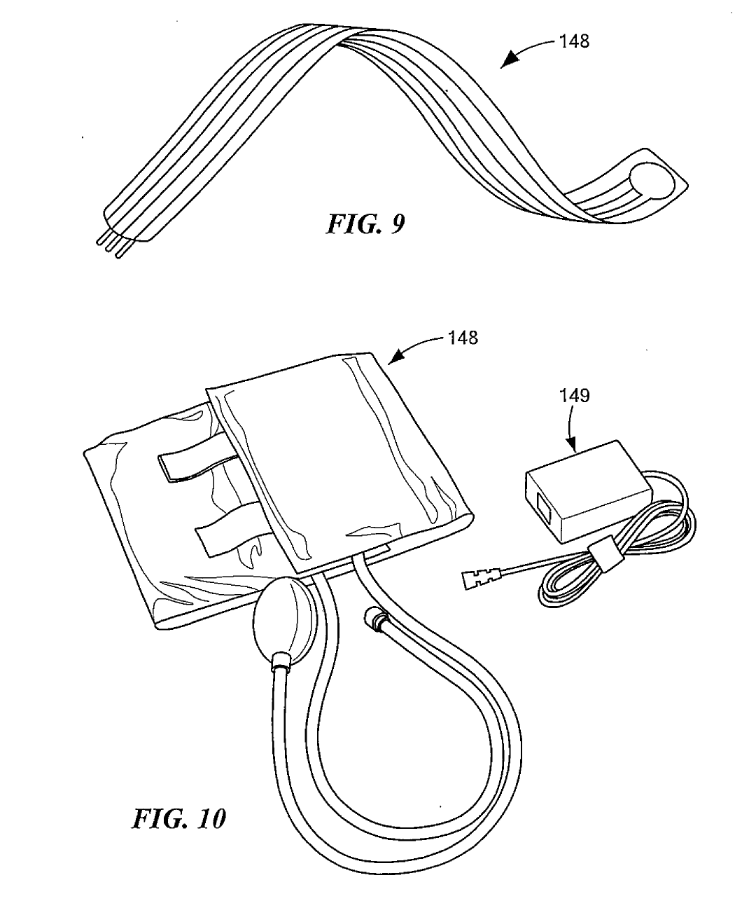

In one example, non-invasive intracranial pressure monitoring system 20, FIG. 2, and the method thereof, FIG. 7, preferably uses the first output signals from first sensor 22, the second output signals from second sensor 24, and the third output signals from third sensor 26 to extract the information needed from the perfusion field of the supraorbital artery, the external carotid artery or one of its branches and the distal artery, as discussed above. The data from the supraorbital artery measured by first sensor 22 may be analyzed with data obtained from an identical second sensor 24 on a perfusion field of the external carotid artery or one of its branches, either on the ear lobe (auricular artery) of ear 25, FIG. 2, e.g., one of locations 106 or 108, FIG. 4, or the temple, e.g., one of locations 110 or 112 (temporal artery). These locations are at a comparable distance from the heart 33, FIG. 2, as supraorbital artery 10, FIG. 1. Therefore, the second output signals from second sensor 24, FIG. 2, can be used to exclude the part of the signal that stems from whole body vascular resistance and pressure. In one example, non-invasive intracranial pressure monitoring system 20, FIG. 2, and the method thereof, FIG. 7, may utilize third sensor 26 placed on the finger or other part of the body, e.g., any of locations shown in FIGS. 5 and 6 discussed above, as a reference for signals from first sensor 22 and second sensor 24. In another example, third sensor 26 may be configured to measure a physiological parameter that includes blood pressure. In this design, third sensor may be a standard blood pressure monitor, such as a blood pressure cuff 148, FIG. 10, which is placed around the upper arm and measures blood pressure and generates the third output signals via blood pressure cuff subsystem 149 which are sent to processing subsystem 30, FIG. 2, as discussed in further detail below.

The result is non-invasive intracranial pressure monitoring system 20 and the method thereof, FIG. 7, non-invasively, accurately, efficiently, effectively, and continuously determines ICP. System 20 is small, robust, light weight and utilizes very little power. In one example, system 20 may be able to run for a full day using 4 AA batteries. Thus, system 20 is portable and can be used in the battlefield, in the field for sports related injuries, or any similar type situation, to provide an accurate measure of ICP or other physiological conditions to determine the extent of brain injury and enable medical care to timely provide the needed care.

In one embodiment, the algorithm for non-invasive intracranial pressure monitoring system 20 and method thereof performed by processing subsystem 30, FIGS. 2 and 7, to determine ICP, are preferably based on relative time lags between the supraorbital artery and the external carotid artery or one of its branches. In this example, first sensor 22, second sensor 24, and third sensor 26, are preferably NIR sensors and provide signals based on the strength of the reflectance of the subtended tissue at the NIR frequency range that increases when a pulse passes through the monitored perfusion bed. Recording this signal optically, using first sensor 22, second sensor 24, and third sensor 26 as NIR sensors proves to be more robust and less sensitive to sensor placement or motion artifact than conventional tonometry-based systems. Similarly, first sensor 22, second sensor 24, and third sensor 26, FIGS. 2-9, may be pressure sensors placed proximate the suborbital artery, the external carotid artery, or one of its branches, and the distal artery as discussed above. In this example, the first, second, and third sensors configured as pressure sensors measure a signal proportional to the amount of blood in the proximate artery, and the first output signals, the second output signals, and the third output signals are similar to the first output signals, the second output signals, and the third output signals measured by the NIR sensor, discussed above, and processed by processing subsystem 30 in a similar manner to determine ICP as discussed herein.

Non-invasive intracranial pressure monitoring system 20 preferably operates on the principle that a less compliant vascular tree propagates a pressure wave faster than a more compliant tree. Increased pressure surrounding the vessels, such as the pressure in the cranium surrounding the internal carotid effectively stiffens the vasculature. Therefore, a pressure wave in the internal carotid will traverse the cranial vault faster than the same wave traveling in the external carotid. The difference between the two may be very small, and in accordance with system 20 and the method thereof, is preferably more robust to compare each to a distal signal provided by third sensor 26, and then compare the two differences.

In other designs, third sensor 26, FIGS. 2 and 7, may be configured to measure blood pressure, e.g., using blood pressure cuff 148 and blood pressure cuff subsystem 149 subsystem 148, FIG. 9, as discussed above, and generate the third output signals. In this example, first sensor 22 is configured to measure pulsations of an artery receiving blood which emanates from the cranial cavity, e.g., the supraorbital artery as discussed above with reference to FIGS. 2 and 3 and generate the first output signal. Second sensor 24 is configured to measure pulsations of an artery which does not receive blood emanating from the cranial cavity, e.g., the external carotid or one of its branches as discussed above with reference to FIG. 4 and generate the second output signal. The waveforms from the first output signals of first sensor 22 and second output signals from sensor 24 are compared. The difference in the timing between the waveforms of the first output signals and the second output signals provides a measure of much greater the pressure in the cranium, i.e. the ICP, is than the blood pressure which is measured by third sensor 26.

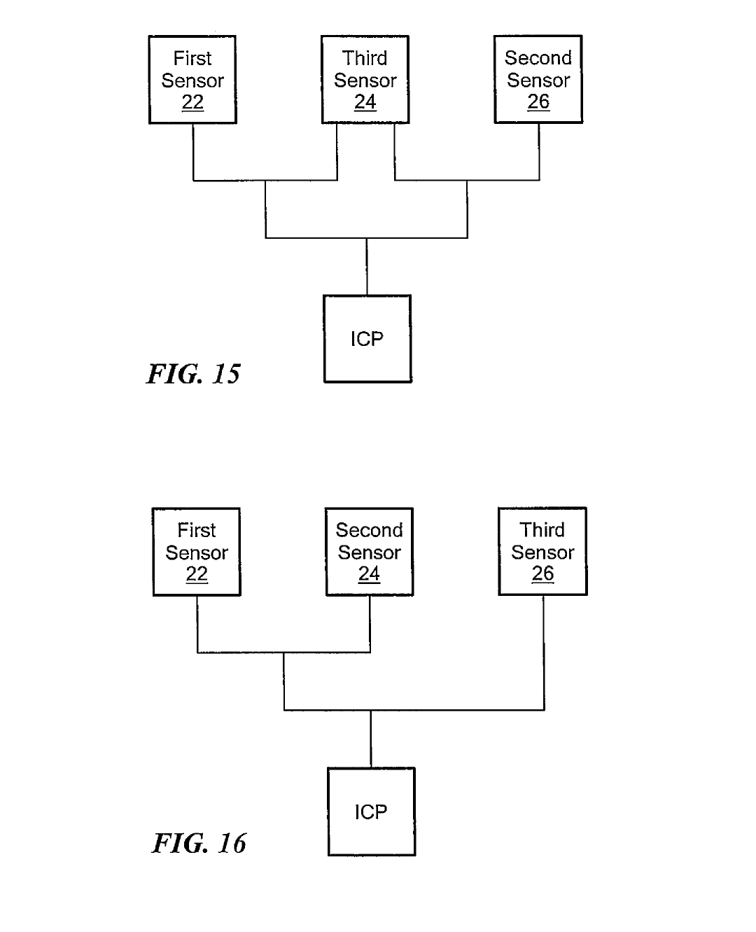

In one embodiment, processing subsystem 30, FIG. 2, and the method thereof, FIG. 7 is configured to determine the ICP by determining the magnitude and phase of the spectral components of the first, second, and third output signals from each of first sensor 22, second sensor 24, and third sensor 26, respectively, e.g., NIR sensors or pressure sensors as discussed above, by comparing the magnitude or the phase of the spectral components of first sensor 22 to the magnitude or the phase of the spectral components of third sensor 26 and the magnitude and phase of the spectral components of second sensor 24 to the magnitude or the phase of the spectral components of third sensor 26 and combining the compared values. In one example, processing subsystem 30 is configured to adjust the value of the component phases according to differences in the magnitudes of the associated spectral components. See FIG. 15 (discussed below).

In another embodiment, processing subsystem 30, FIG. 2, and the method thereof, FIG. 7 is configured to determine the ICP by correlating signals from first sensor 22 to signals from third sensor 26 and correlating signals from second sensor 24 to third sensor 26 and combining the determined correlations. See FIG. 15 (discussed below).

In yet another embodiment, processing subsystem 30 is configured to determine the intracranial pressure by combining signals from first sensor 22 with signals from second sensor 24 and combining that result with signals from third sensor 26. See FIG. 16 (discussed below).

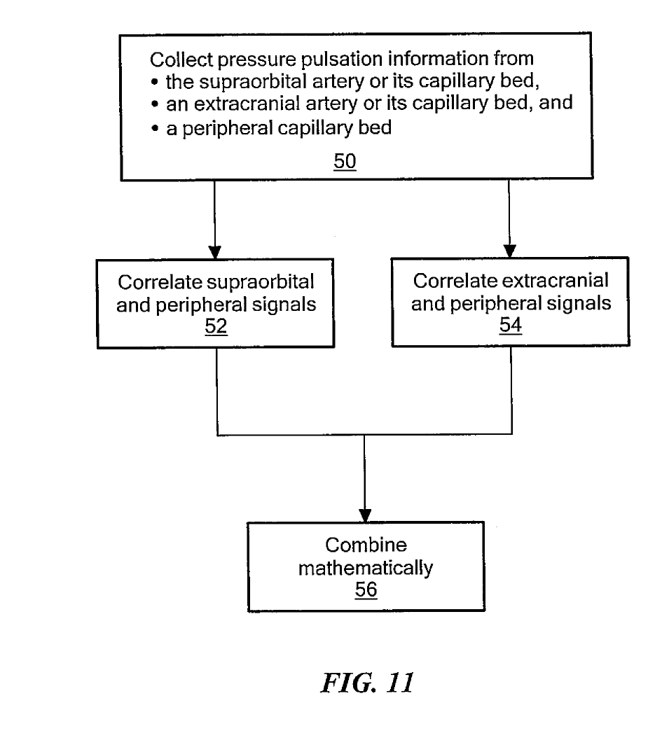

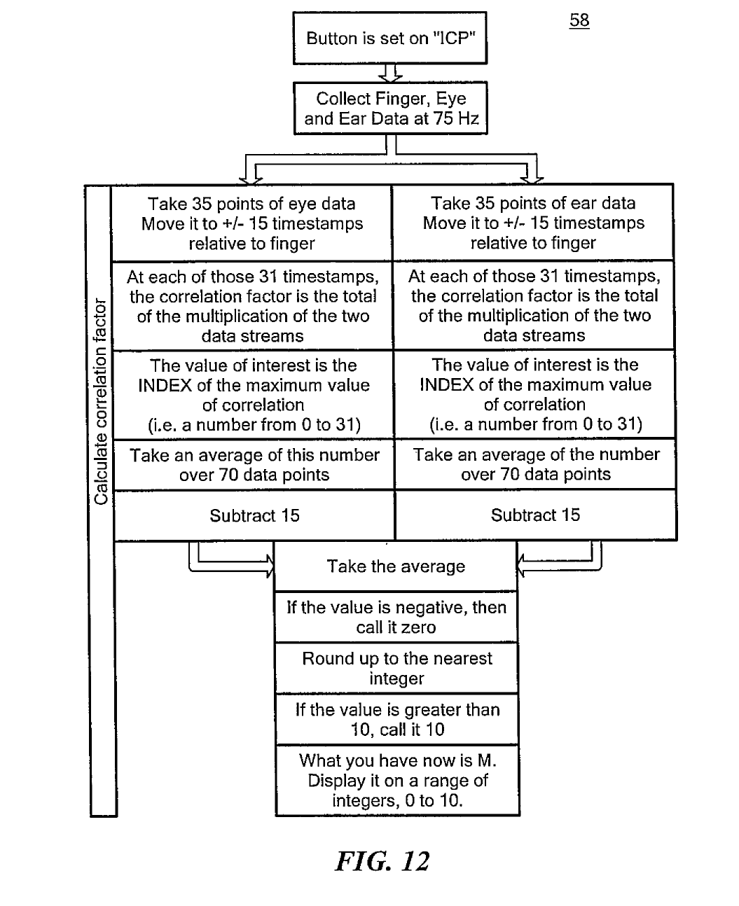

FIG. 11 shows one example of a flowchart of one embodiment of the method of determining ICP using non-invasive intracranial pressure monitoring system 20, FIGS. 2 and 7. In this example, pulsations of the supraorbital artery 10, FIG. 1, are measured by first sensor 22, FIG. 2 placed on forehead 30 at one of location 102 or 104, FIG. 3, pulsations of external carotid artery or one of its branches are measured by second sensor 24 placed at one of locations 106, 108, 110, or 112, FIG. 4. In this example, the one or more physiological parameters measured by third sensor 26 include pulsations of distal artery measured at any of locations 116-144, FIGS. 5 and 6, step 50, FIG. 11. First sensor 22, second sensor 24, and/or third sensor 26 may also measure pressure as discussed above to generate the first output signals, the second output signals, and the third output signals which are processed similarly. Signals from first sensor 22 to the third sensor 26 are correlated, step 52. Signals from second sensor 24 and the third sensor 26 are then correlated, step 54. The signals from steps 52 and 54 are combined mathematically to determine ICP, step 56. Flow chart 58, FIG. 12 shows a more detailed specific implementation of one example of the method shown in FIGS. 7 and 11.

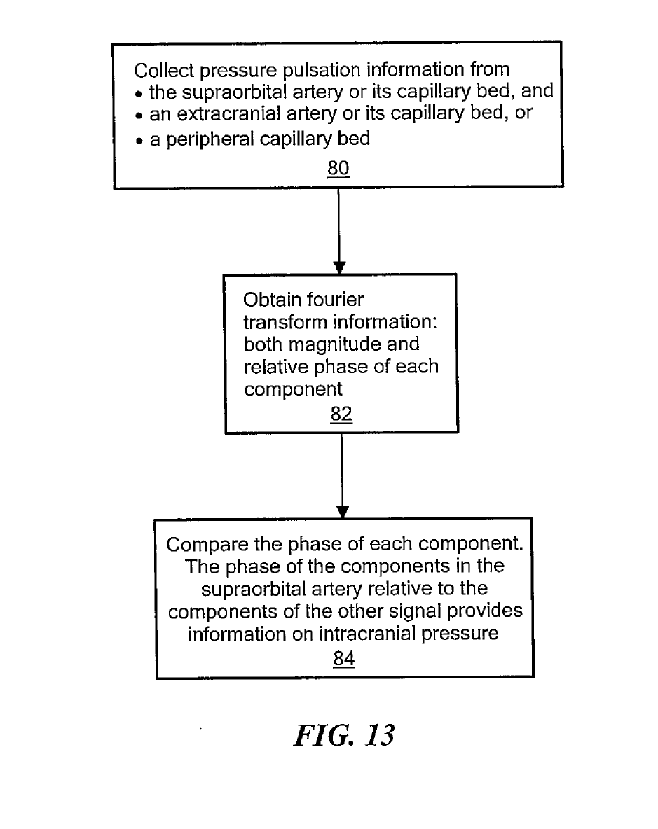

FIG. 13 shows an example of a flowchart of another embodiment of the method of determining ICP using non-invasive intracranial pressure monitoring system 20, FIGS. 2 and 7. In this example, pulsations of the supraorbital artery 10, FIG. 1, are measured by first sensor 22, FIG. 2, placed on forehead 30 at one of locations 102 or 104, FIG. 3, which generates the first output signals. Pulsations of external carotid artery 18 or one of its branches are measured by second sensor 24 of one of locations 106, 108, 110 or 112, FIG. 4. Pulsation of the distal artery are measured by third sensor 26 placed proximate at one of locations 114-144, FIGS. 5 and 6, step 80, FIG. 13. First sensor 22, second sensor 24, and/or third sensor 26 may also measure pressure as discussed above to generate the first output signals, the second output signals, and the third output signals. Processing subsystem 30, FIG. 2, responsive to the first output signals from first sensor 22, the second output signals from sensor 24, and the third signals from sensor 26, performs a Fourier transform to determine the magnitude and phase of spectral components of the first, second, and third output signals from each of first sensor 22, second sensor 24, and third sensor 26, step 82. The phase of the spectral components of first sensor 22 is compared to the phase of the spectral components of third sensor 26 and the phase of the spectral components of second sensor 24 is compared to the phase of the spectral components of third sensor 26, and the values are combined to determine ICP, step 84. See FIG. 15. Preferably, processing subsystem 30, FIGS. 2 and 7, is configured to adjust the value of the component phases according to differences in magnitudes of associated spectral components.

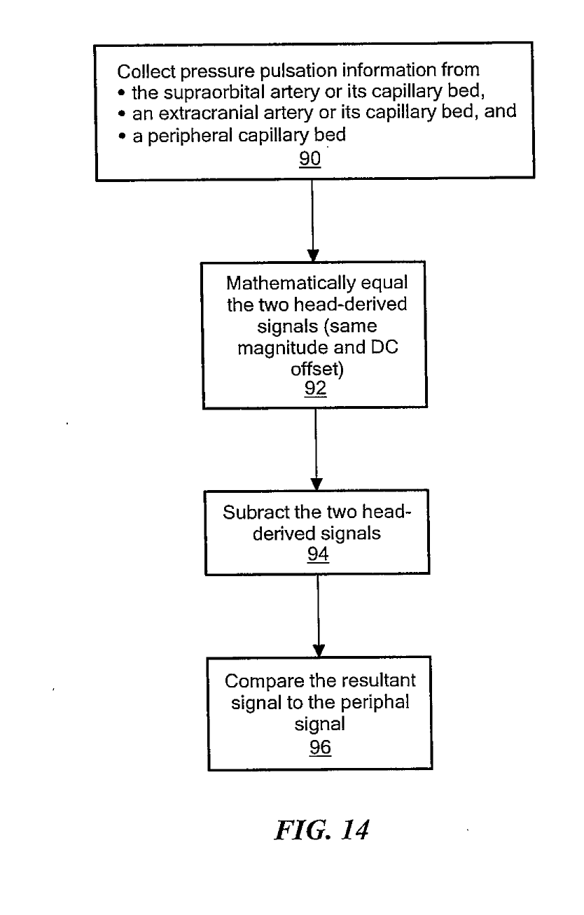

FIG. 14 shows a flowchart of another embodiment of the method of determining ICP using non-invasive ICP monitoring system 20, FIGS. 2 and 7. In this example, pulsations of the supraorbital artery 10, FIG. 1, are measured by first sensor 22, FIG. 2, placed on forehead 30 and one of locations 102 or 104, FIG. 3, and pulsations of external carotid artery 18 or one of its branches are measured by second sensor 24 placed at one of locations 106, 108, 110, or 112, FIG. 4. In this example, pulsations of distal artery are measured by third sensor 26 at one of locations 114-144, FIGS. 5 and 6, step 90, FIG. 14. First sensor 22, second sensor 24, and/or third sensor 26 may also measure pressure as discussed above to generate the first output signals, the second output signals, and the third output signals which are processed similarly. Processing subsystem 30, FIGS. 2 and 7, is configured to determine ICP by combining signals that are mathematically equal in at least one mathematical measure, such as offset value or maximum value from first sensor 22 with signals from second sensor 24, step 92. The result of step 92 is combined with signal from third sensor 26, step 96. See FIG. 16.

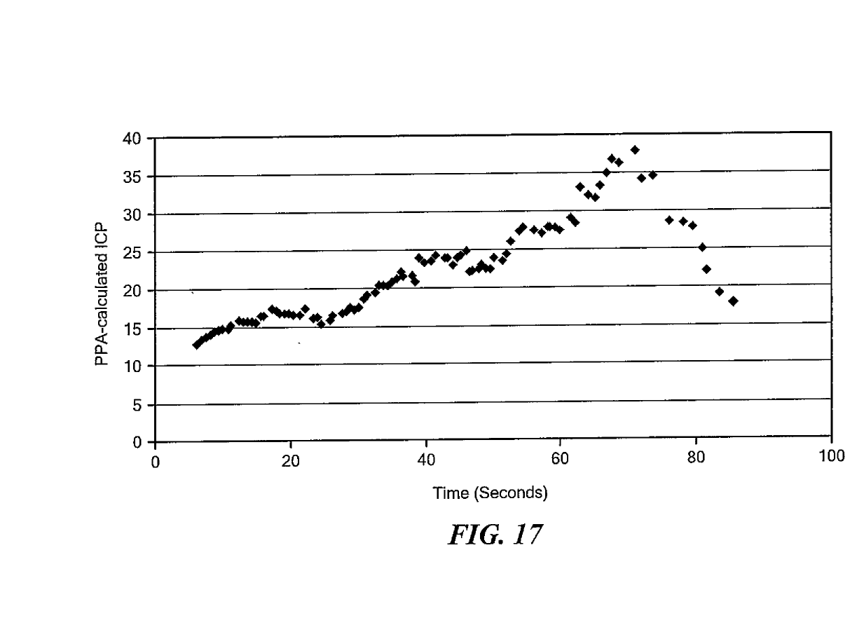

An initial demonstration of the non-invasive intracranial pressure monitoring system 20, and method thereof shown in one or more of FIGS. 2-16, was conducted in an animal test. This test was used to verify that the ovine model was appropriate for the test and that non-invasive intracranial pressure monitoring system 20 and the method thereof, can obtain the necessary data for calculating a measure of ICP. This early prototype utilized a laptop computer to acquire data from the first sensor 22, second sensor 24, and third sensor 26. The promising results are shown in FIG. 17.

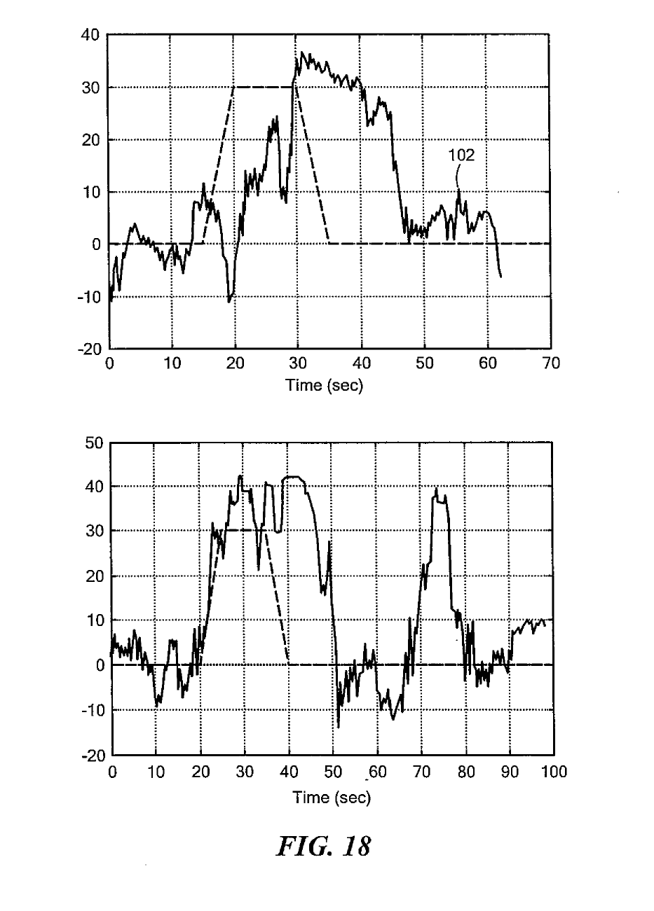

With the preliminary ovine model completed, non-invasive intracranial pressure monitoring system 20 was further tested. The intracranial pressure of a subject was artificially increased due to hydrostatic pressure present in tilt from horizontal to upside down. FIG. 18 shows two such results from different subjects. Curve 102 indicates the tilt of the chair, from horizontal (zero) to upside down (recorded as 30). The value of 30 was assigned to the chair tilt as it is approximately the expected increase in the ICP, in cmH20, due to hydrostatic pressure. In the pilot study on healthy subjects, the exact value of the increase in ICP is unknown, and so the ICP algorithm was scaled by this value of 30 cm H20 across the data from all 6 subjects. In the second image shown in FIG. 18 (on the bottom), the inversion chair did not home properly and underwent a second, more rapid, inversion. Non-invasive intracranial pressure monitoring system 20 and the method thereof was able to determine the resultant increase in ICP in both excursions with high fidelity as seen in the image.

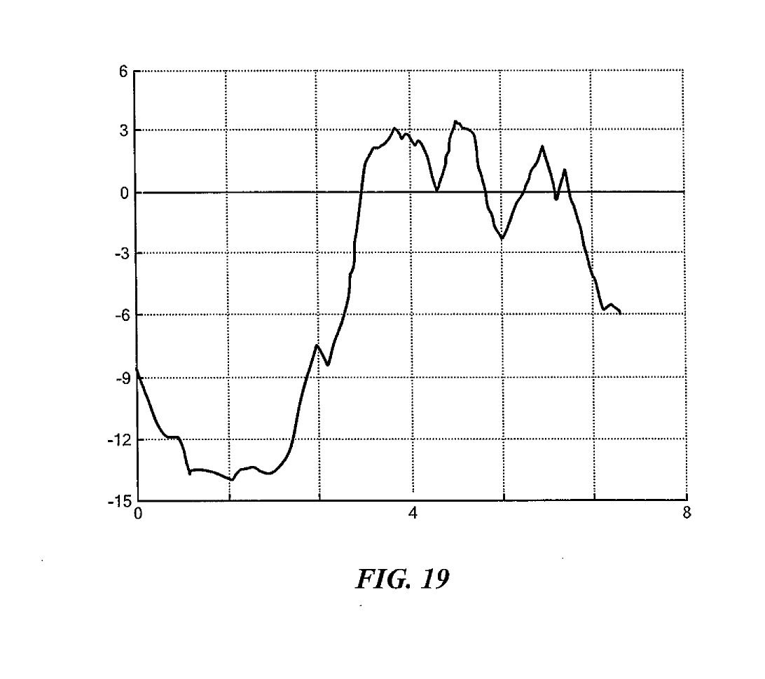

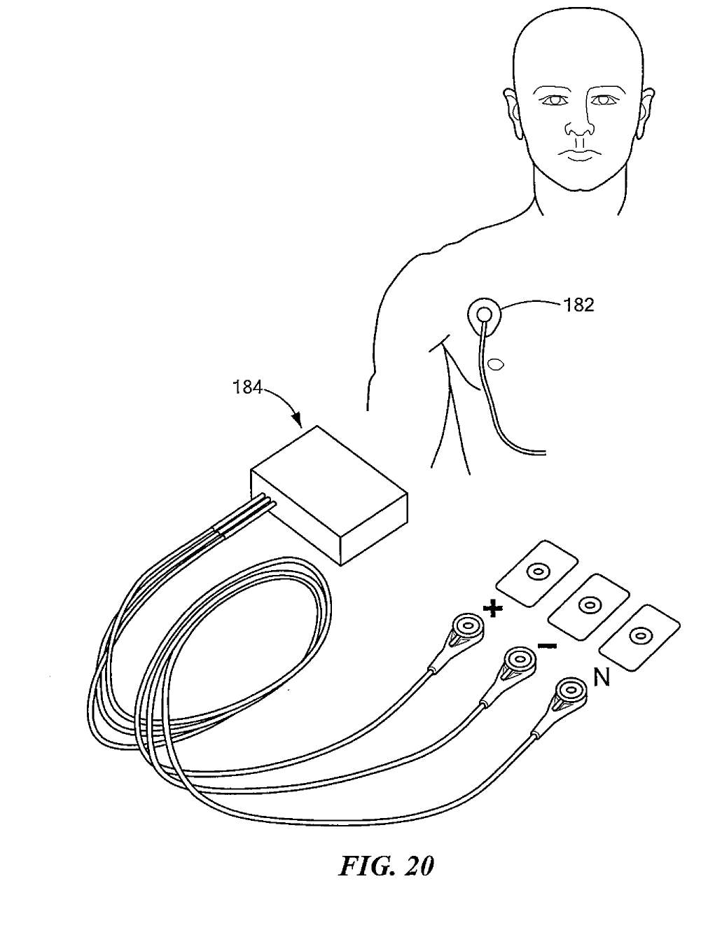

In a separate experiment, non-invasive intracranial pressure monitoring system 20 and the method thereof, shown in one or more of FIGS. 2-16, was used to record data during a squat-to-stand test (2 minutes of squat to straight standing). Non-invasive intracranial pressure monitoring system and the methods thereof discussed above was able to determine the negative value of ICP that is expected with such a test. The results are shown in FIG. 19. As shown, immediately after the subject stands, TCP drops to below the normal level, indicated as “zero”, and rises back to normal within four seconds, oscillating about the normal value for the duration of the test.

In another design, one or more of first sensor 22, second sensor 24, and/or third sensor 26 of system 20, FIG. 2, and the method thereof, FIG. 7, may be configured to measure electrical signals, e.g., an electrocardiogram signal using electrocardiogram sensor 182, FIG. 20, coupled to electrocardiogram subsystem 184, or similar type sensor which measures electrical signals of the human body. For example, system 20, FIG. 2, may include first sensor 22 which is configured to monitor pulsations of an artery receiving blood which emanates from the cranial cavity, e.g., the supraorbital artery at one of locations 102 or 104, FIG. 3, as discussed above, and generate the first output signals. Second sensor 24 may be placed proximate to a profusion field of an artery which does not receive blood emanating from the cranial cavity, e.g., the external carotid artery or one of its branches at one of locations 106, 108, 110, or 112, FIG. 4 as discussed above, and generate the second output signals. Third sensor 26 may be configured to record an electrocardiogram signal using electrocardiogram sensor 182, FIG. 20 and electrocardiogram subsystem 184 generates the third output signals. The electrocardiogram signal measured by third sensor 26 may be used to provide the timing of the heartbeat of a human subject. The ICP is then calculated as the difference between the two signals measured by the first sensor 22 and the second sensor 24 over the pressure determined due to the lag from the signals recorded by the first sensor 22 and second sensor 24, as discussed below.

Processing subsystem 30, FIG. 2, and the method thereof, FIG. 7, is preferably configured to determine the intracranial pressure by determining a first lag time between a peak signal of a signal from the first output signals to a peak of a signal from the third output signals to a second lag time peak between a signal from the second output signals to a peak of a signal from the third output signals and calculating the intracranial pressure based on the difference between the first lag time and the second lag time. Processing subsystem 30 may also be configured to determine intracranial pressure by determining a lag time between a peak of a signal from the first output signal to a peak of a signal from the second output signal and calculating the intracranial pressure based on the time lag. In yet another example, processing subsystem 30 may be configured to determine intracranial pressure by determining a first lag time between a peak of a signal from the first output signal to a peak of a signal from the third output signals, a second lag time between a peak of a signal from the second output signals to a peak of signal from the third output signals and a third lag time between the peak of the signal from the first output signals to a peak of the signal from the second output signals and calculating the intracranial pressure based on the differences of the first, second, and third time lags.

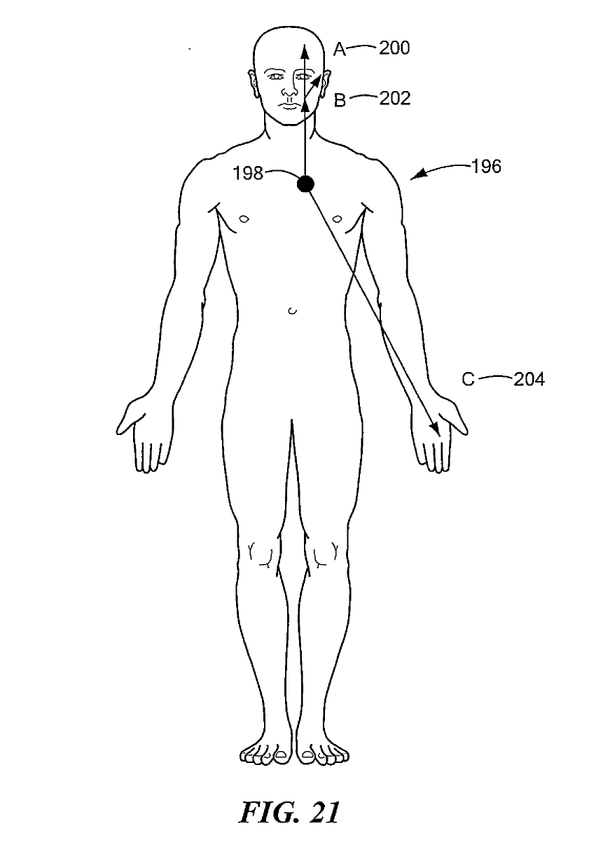

For example, as shown in FIG. 21, there is a time delay for the pulsation signals measured by first sensor 22, second sensor 24, and third sensor 26 to reach different points on human body 196, from point 198, e.g., the location proximate the heart of human subject Time A-200 is the time for a signal to reach the supraorbital artery, e.g., from point 198. Time B-202 is the time for a signal to reach the external carotid artery or one of its branches from point 198. Time C-204 is the time for a signal to reach a distal artery from point 198. When third sensor 26 is configured as an electric sensor, e.g., as electrocardiogram sensor 182, FIG. 20, then time to reach C-204, FIG. 21, is 0. Time A-200, B-202, and C-204 depend on the stiffness of the blood vessels of human subject 196, FIG. 21. The stiffness of the blood vessels to points B-202 and C-204 depends on the physiology of human subject 198 including their height, weight, and presence of arteriosclerosis. The stiffness of the blood vessels to point A-200 depends on these factors and also on ICP.

In simplest terms, the ICP, P, is linearly related to the time delay A-200 less some part of B-202. However, the exact amount of B that should be subtracted from A is unknown. The delay to C-204 is used to approximate the amount of B-202 that should be subtracted from A-200, so that with weights M and N, we can calculate P as follows:

P=M(A−C)+N(B−C) (1)

where the weights M and N are determined by taking a known set of P, A-200, B-202, and C-204 and calculating the weights M and N that result in the smallest error for this equation.

Adding in greater complexity to account for known effects of the arterial tree on the propagation of the wave, not only the time delay of the bulk of the pulse is analyzed, but also on the relative delays of different frequency components of the pulse. The analysis proceeds in the same manner, with greater granularity achieving lower errors. For example, with Aₒ-200, Bₒ-202, and Cₒ-204 indicating the timing of the main wave, determined by correlation, and Aₓ, Bₓ, and Cₓ indicating the timing of the frequency component at X Hz, ICP can be determined by the equation:

P=Mₒ(Aₒ−Cₒ)+Nₒ(Bₒ−Cₒ)+Mₓ(Aₓ−Cₓ)+Nₓ(Bₓ−Cₓ) (2)

for any number of X. Equation (2) is solvable for all constants to determine ICP provided a large enough set of known data.

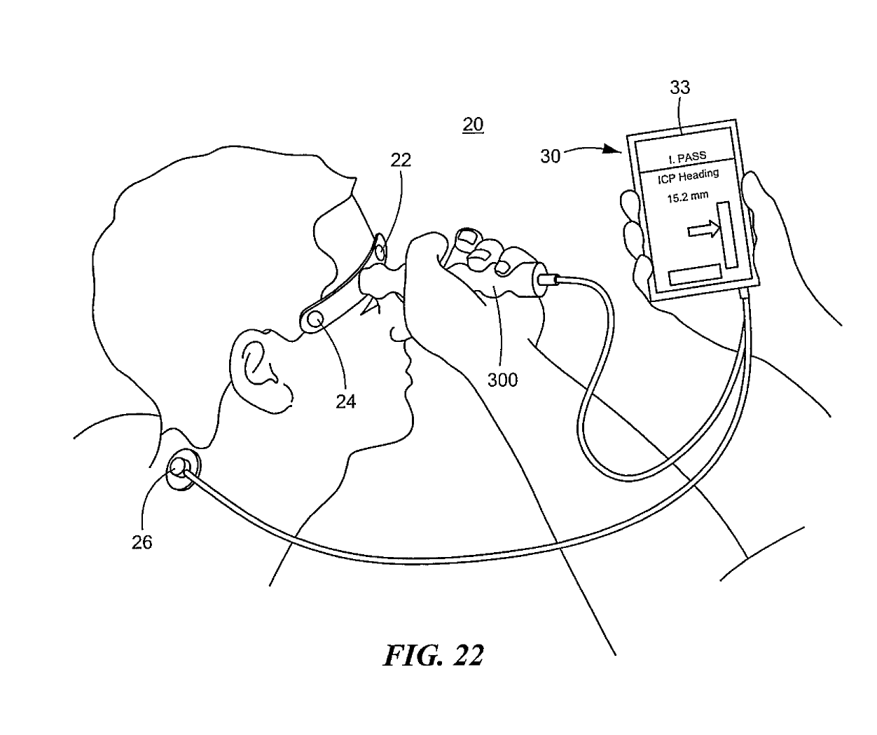

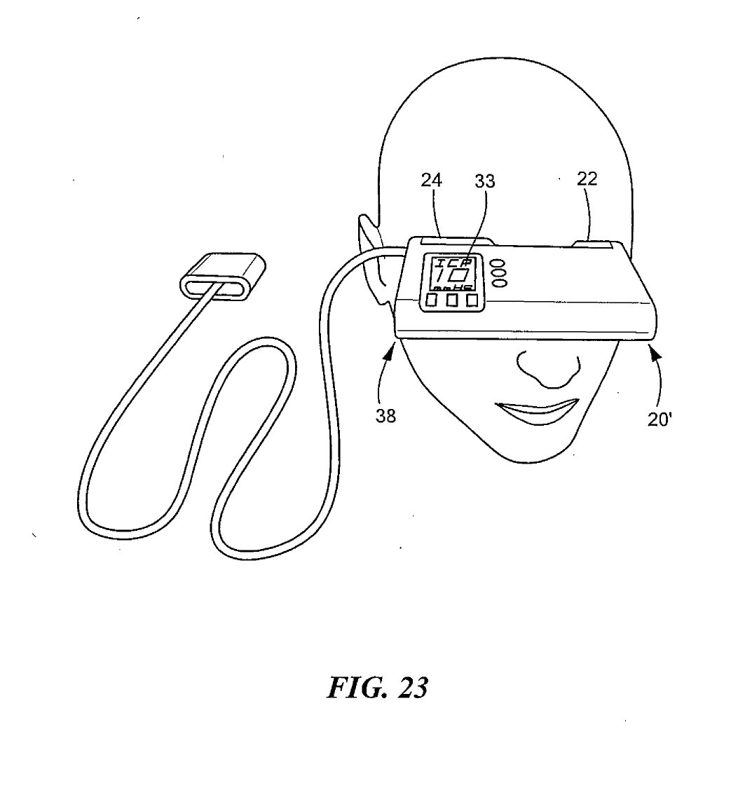

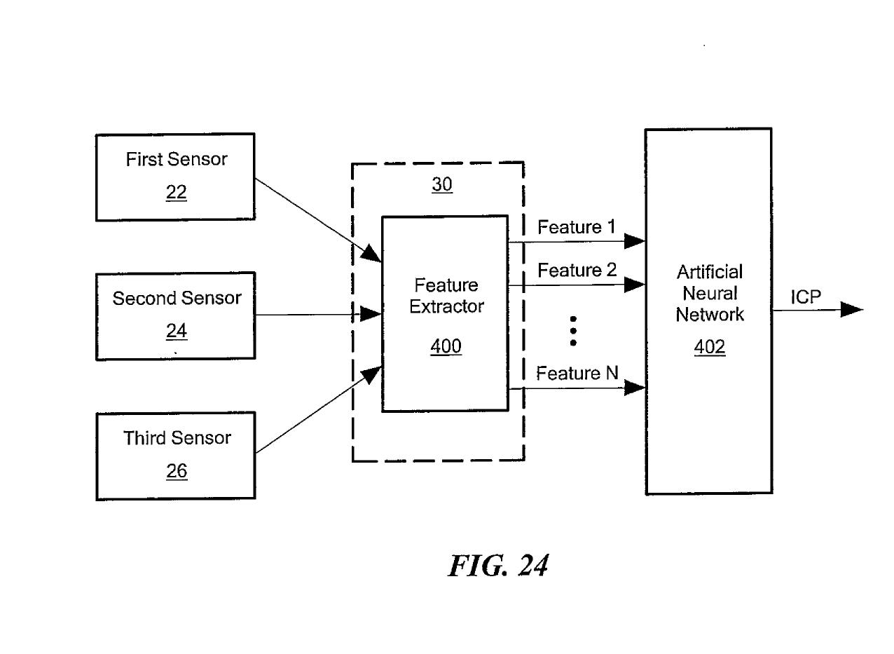

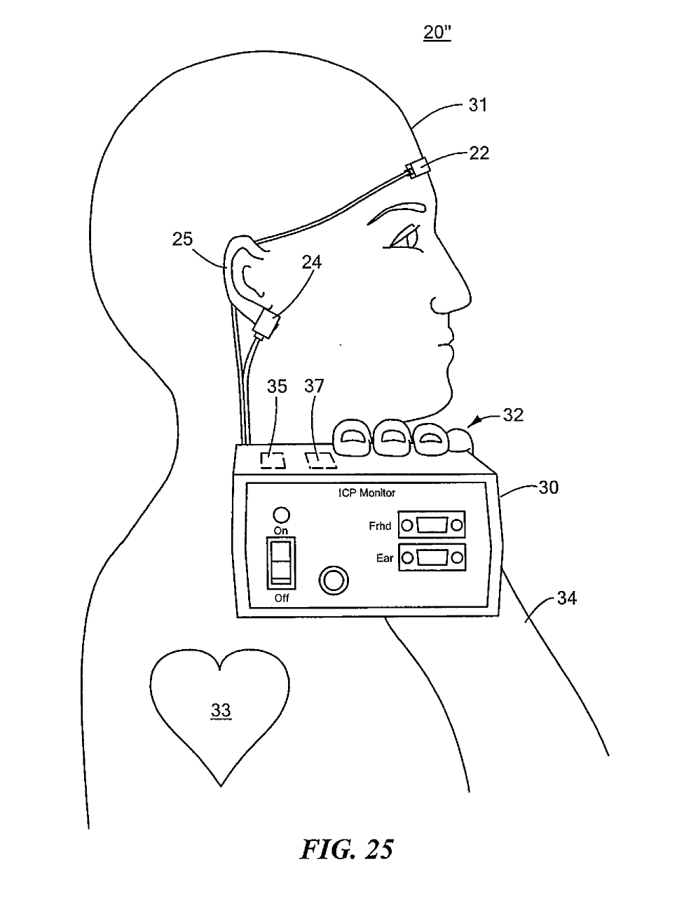

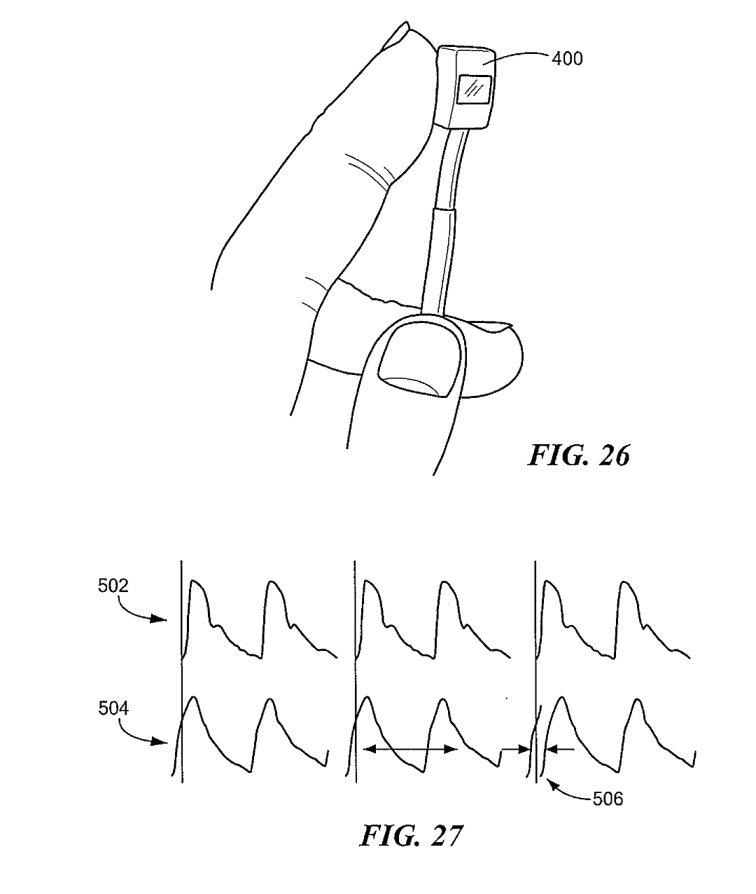

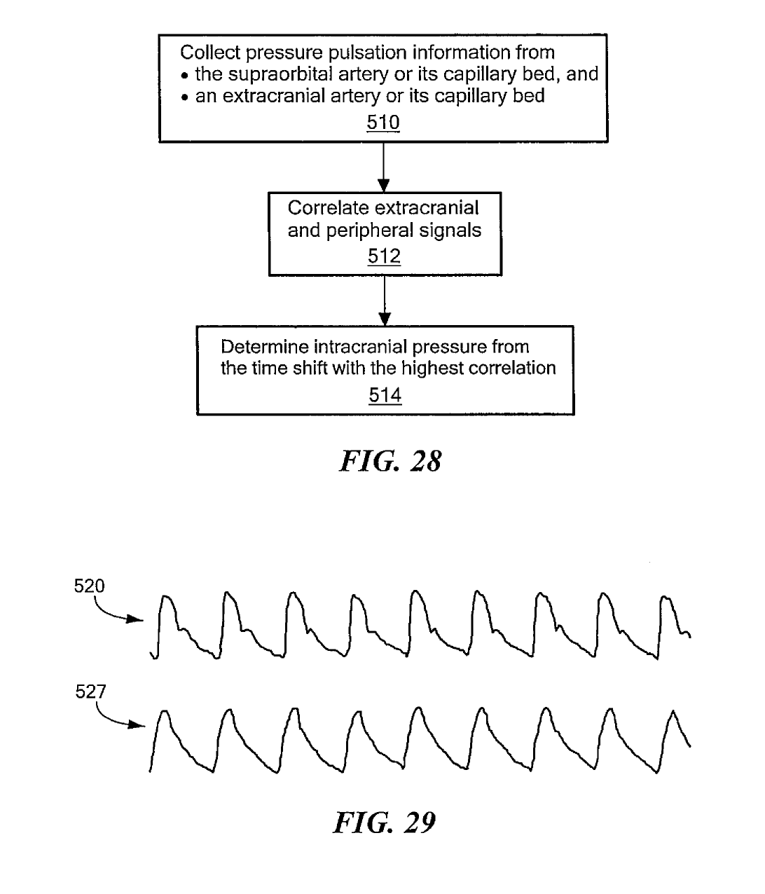

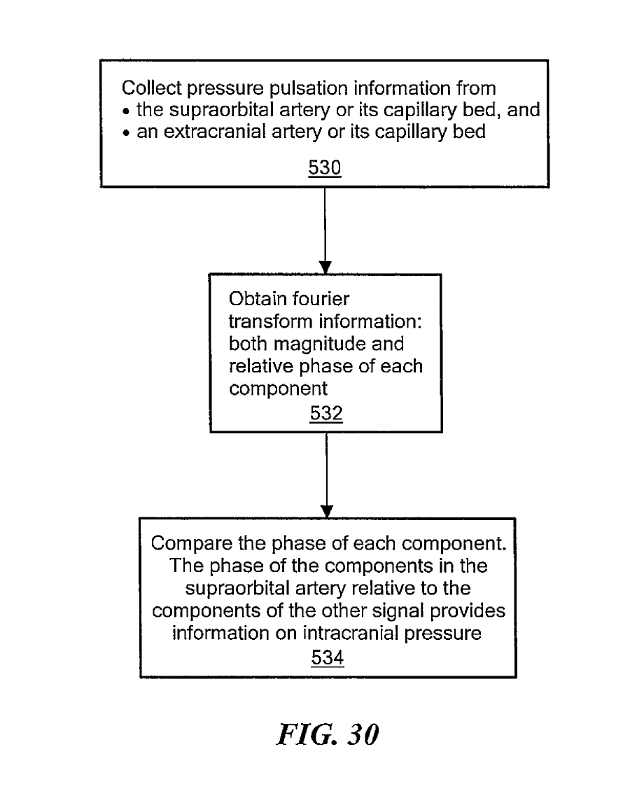

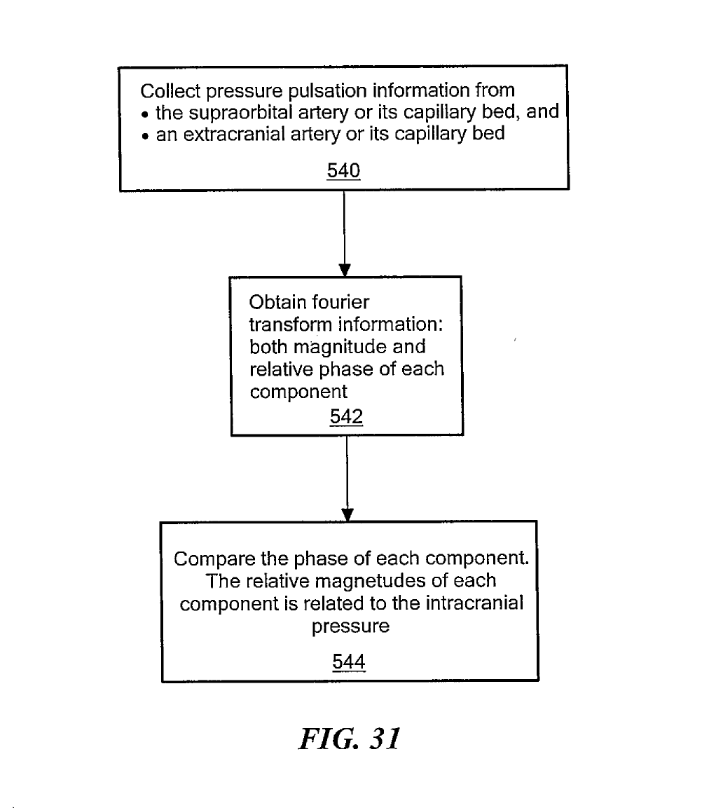

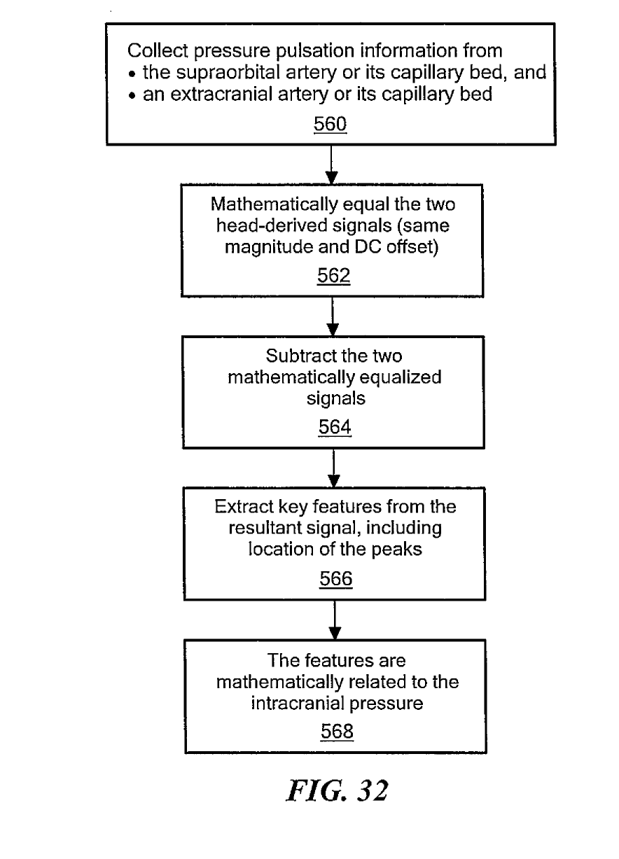

In one example, system 20 shown in one or more of FIGS. 2-21, may include hand-held device 300, FIG. 22, configured to hold first sensor 22 and second sensor 24 in a spaced orientation as shown, such that first sensor 22 is placed proximate the profusion field of an artery receiving blood which emanates from the cranial cavity, e.g., the supraorbital artery as discussed above, e.g., at locations 102 or 104, FIG. 3, and second sensor is placed proximate the artery which does not receive blood emanating from the cranial cavity, e.g., the external carotid artery or one of its branches at one of locations 106, 108, 110, or 112, FIG. 4, e.g., as shown in FIG. 22. In this example, third sensor 26 is placed behind the neck as shown.

System 20, and the method thereof shown in one or more of FIGS. 2-20, with first sensor 22, second sensor 24, and processing subsystem 30, may be integrated as hand-held system 20′, FIG. 23, as shown.

System 20 and the method thereof shown in one or more of FIGS. 1-23 may also be configured to determine another physiological condition such as a stroke. For example, system 20 and the method thereof may be of particular use when a patient is suspecting of having suffered a stroke. There are two types of strokes: ischemic and hemorrhagic. In an ischemic stroke, a blood clot or other obstruction in a blood vessel prevents blood from reaching parts of the brain. In a hemorrhagic stroke, blood exits the arterial tree, building up in the brain. Although the initial outward symptoms are very similar, the recommended course of treatments are different. Blood thinners may help a patient suffering from an ischemic stroke, however they will cause more harm to one suffering from a hemorrhagic stroke. A hemorrhagic stroke, however, will cause an increase in the intracranial pressure, while an ischemic stroke does not. System 20 and the method thereof disclosed herein can be very valuable for this patient population, as a measurement of ICP on a patient suffering from a stroke can more assuredly be provided with an appropriate treatment.

In one embodiment, processing subsystem 30, FIG. 2, may include feature extractor 400, FIG. 24, configured to calculate one or more features from one or more of the first output signals, the second output signals 24, and/or the third output signals generated by first sensor 22, second sensor 24, and third sensor 26, and output the one or more features to artificial neural network 402 which is configured to calculate ICP based on the one or more features. The one or more features calculated by feature extractor may include one or more of an amplitude of a largest peak in one of the first output signals, the second output signals and/or the third output signals, a time from a beginning of a data acquisition cycle to a time of a maximum peak of one of the first output signals, the second output signals and/or the third output signals, and a time between two different peaks of one of the first output signals, the second output signals and/or the third output signals.

Feature extractor 400 is preferably configured to calculate the one or more features from a combination of signals from the first output signals, the second output signals, and/or the third output signals. The one or more features may include one or more of a time from a peak of one of the first output signals, the second output signals and/or the third output signals to a corresponding time peak of another of the first output signals, the second output signals and/or the third output signals, and a difference of a magnitude of a peak of one of the first output signals, the second output signals and/or the third output signals, to a magnitude of the peak of another of the first output signals, the second output signals, and/or the third output signals.

Preferably, artificial neural network 402 is configured to combine the one or more features in a non-linear fashion based on various weights and the structure of artificial neural network to provide the intracranial pressure.

System 20 and the method thereof shown in one or more of FIGS. 1-24 discussed above may be implemented by computer program instructions. These computer program instructions may be provided to one or more of processors, a general purpose computer, a special purpose computer, or other programmable data processing apparatus to produce a machine, such that the instructions, which execute via the one or more processors create means for implementing the functions/acts specified in the flowchart and/or block diagram block or blocks shown in one or more of FIGS. 1-24.

System 20, 20′, 20″ and the method thereof shown in one or more of FIGS. 1-32 discussed above may be implemented by computer program instructions. These computer program instructions may be provided to one or more of processors, a general purpose computer, a special purpose computer, or other programmable data processing apparatus to produce a machine, such that the instructions, which execute via the one or more processors create means for implementing the functions/acts specified in the flowchart and/or block diagram block or blocks shown in one or more of FIGS. 1-32.

For enablement purposes only, the following code portions are provided which can be executed on one or more processors 35, FIGS. 2 and/or 25, of processing subsystem 30, or a computer to carry out the primary steps and/or functions of system 20 and the method thereof discussed above with reference to one or more of FIGS. 1-32 and recited in the claims hereof. Other equivalent algorithms and code can be designed by a software engineer and/or programmer skilled in the art using the information provided herein.

Although discussed this far with reference to one or more of FIGS. 1-24, system 20, 20′ includes first sensor 22, second sensor 24, and third sensor 26, in another embodiment, system 20″, FIG. 25, where like parts have been given like numbers, does not include third sensor 26. In this embodiment, system 20″ similarly includes first sensor 22 placed proximate a diffusion field of an artery of a human subject receiving blood which emanates from within cranial cavity 14, FIG. 1, and generates the first output signals. In one example, first sensor 22 is placed proximate supraorbital artery 10, the supratrocheal artery (not shown) or the ophthalmic artery (not shown) when first sensor 22 is placed on forehead 30 or at one of locations 102 or 104, FIG. 3. Similarly, second sensor 24 is placed proximate a profusion field of an artery which does not receive blood emanating from cranial cavity (an extracranial artery) and is configured to measure pulsations of that artery and generates the second output signals. As discussed above, second sensor 24 may be placed proximate ear 25, FIG. 25, e.g., as shown on the earlobe which is near external carotid artery 18 or one of its branches at one of locations 106 or 108, FIG. 4. In other examples, second sensor 24, FIG. 25, may be placed on or near the temple, e.g., at one of locations 110 or 112, FIG. 4. In one example, first sensor 22 and second sensor 24 are preferably placed approximately the same distance from heart 33. First sensor 22, FIG. 25, and second sensor 24 may be configured as (NIR) sensors as discussed above with reference to at least FIG. 8, pressure sensors as discussed above with reference to FIG. 9, or as photoplethysmograph sensors, e.g., photoplethysmograph sensor 400, FIG. 26, e.g., available from Nonin Inc, Plymouth Minn., or as made by

Vivonics Inc., Sudbury Mass. First sensor 22 and second sensor 24 may also be a combination of an NIR sensor, a pressure sensor, and/or a photoplethysmograph sensor.

In this embodiment, processing subsystem 30, FIG. 25, is responsive to the first output signals from first sensor 22 and the second output signals from second sensor 24 and is configured to calculate a cross-correlation of the first output signals and the second output signals and determine the intracranial pressure of the human subject from a time shift of the cross-correlation with the highest value. For example, in signal processing, cross-correlation is a measure of the similarity of two series as a function of the lag of one relative to the other. This is known as a sliding dot product or sliding inner product. FIG. 27 shows representative first output signals 502 and second output signals 504 generated by first sensor 22 and second sensor 24, FIG. 25. One of the first output signals or the second output signals is moved along the time axis, e.g., in this example, second output signal 504 as shown. The cross-correlation is calculated by processing subsystem 30 at each point by multiplying the values at each point and summing the products. The point where the cross-correlation is highest, e.g., indicated at 506, is used to determine ICP. Thus, system 20″, FIG. 25, effectively, efficiently, and non-invasively determines ICP using only first sensor 22, second sensor 24 and processing system 30.

FIG. 28 shows one example of a flowchart of the corresponding method for this embodiment of non-invasively measuring ICP for system 20″, FIG. 25. In this example, pressure pulsation information is collected from the supraorbital artery or its capillary bed, the supratrocheal artery, or the ophthalmic artery using first sensor 22 and the first output signals are generated, step 510, FIG. 28. Similarly, pressure pulsation information is collected from an extracranial artery or its capillary bed, e.g., the external carotid artery or one of its branches, using second sensor 24 and the second output signals are generated, as discussed above with reference to FIG. 25, step 510, FIG. 28. The extracranial and peripheral signals of the first output signals and the second output signals are then correlated, step 512. ICP is determined from the time shift of the cross-correlation with the highest value, step 514.

In another embodiment, processing system 30, FIG. 25, of system 20″ may be configured to calculate a phase shift of different frequencies included in the first output signals and the second output signals and processing system 30 determines ICP of the human subject from the phase shift at different frequencies of the first output signals and the second output signals. FIG. 29 shows exemplary first output signals 520 generated by first sensor 22 and second output signals generated by second sensor 24 used to determine the intracranial pressure of the human subject from the phase shift at different frequencies of the first output signals and the second output signals.

FIG. 30 shows an example of a flowchart of this embodiment of the method for non-invasively determining ICP using first output signals 520, FIG. 29, and second output signals 522. In this example, pulsations of the supraorbital artery 10, FIG. 1, the supratrocheal artery, or the ophthalmic artery are measured by first sensor 22 as discussed above with reference to FIG. 25 to generate first output signals 520, FIG. 29, step 530, FIG.

30. Pulsations of external carotid artery 18 or one of its branches are measured by second sensor 24 as discussed above with reference to FIG. 25 to generate the second output signals, step 530. Processing subsystem 30, FIG. 25, responsive to first output signals 520, FIG. 29, and second output signals 522 performs a Fourier transform to determine the magnitude and phase of spectral components of first output signals 520 and second output signals 522, step 532, FIG. 30. The phase of the spectral components of first output signals 520 is compared to the phase of the spectral components of second output signals 522 to determine ICP, step 534. Preferably, processing subsystem 30, FIG. 25, is configured to adjust the value of the component phases according to differences in magnitudes of associated spectral components.

In another embodiment, processing subsystem 30, FIG. 25, of system 20″ may be configured to calculate a magnitude of different frequencies included in first output signals 520, FIG. 29 and second output signals 522 and processing system 30 determines intracranial pressure of the human subject from a difference in magnitudes of the different frequencies of the first output signals and the second output signals.

FIG. 31 shows an example of a flowchart of this embodiment of the method for non-invasively determining ICP using first output signals 520, FIG. 29 and second output signals 522. In this example, pulsations of the supraorbital artery 10, FIG. 1, the supratrocheal artery, or the ophthalmic artery are measured by first sensor 22 as discussed above with reference to FIG. 25 to generate first output signals 520, FIG. 29, step 540, FIG. 31. Pulsations of external carotid artery 18 or one of its branches are measured by second sensor 24 as discussed above with reference to FIG. 25 to generate second output signals 522, FIG. 29, step 540. Processing subsystem 30, FIG. 25, responsive to first output signals 520 and second output signals 522 performs a Fourier transform to determine the magnitude and phase of spectral components of first output signals 520 and second output signals 522 step 542, FIG. 31. The magnitude of the spectral components of first output signals 520 is compared to the magnitude of the spectral components of second output signals 522 to determine ICP, step 544. Preferably, processing subsystem 30, FIG. 25, is configured to adjust the value of the component phases according to differences in magnitudes of associated spectral components.

In yet another embodiment, processing subsystem 30, FIG. 25, of system 20″ may be configured to calculate a difference between first output signals 520, FIG. 29 and second output signals 522 and processing system 30 determines ICP from the difference.

FIG. 32 shows an example of a flowchart of this embodiment of the method of non-invasively determining ICP. In this example, pulsations of the supraorbital artery 10, FIG. 1, the supratrocheal artery, or the ophthalmic artery are measured by first sensor 22 as discussed above with reference to FIG. 25 to generate first output signals 520, FIG. 29, step 560, FIG. 32. Pulsations of external carotid artery 18 or one of its branches are measured by second sensor 24 as discussed above with reference to FIG. 25 to generate second output signals 522, FIG. 29, step 560. Processing subsystem 30, FIG. 25, responsive to first output signals 520 and second output signals 522 is configured to determine ICP by combining signals that are mathematically equal in at least one mathematical measure, such as offset value or maximum value from first sensor 22 with signals from second sensor 24, step 562, FIG. 32. The two mathematically equal signals are subtracted, step 564. Key features are extracted from the resultant signal, step 566. The features are used to determine ICP, step 568.

Although specific features of the invention are shown in some drawings and not in others, this is for convenience only as each feature may be combined with any or all of the other features in accordance with the invention. The words “including”, “comprising”, “having”, and “with” as used herein are to be interpreted broadly and comprehensively and are not limited to any physical interconnection. Moreover, any embodiments disclosed in the subject application are not to be taken as the only possible embodiments.

In addition, any amendment presented during the prosecution of the patent application for this patent is not a disclaimer of any claim element presented in the application as filed: those skilled in the art cannot reasonably be expected to draft a claim that would literally encompass all possible equivalents, many equivalents will be unforeseeable at the time of the amendment and are beyond a fair interpretation of what is to be surrendered (if anything), the rationale underlying the amendment may bear no more than a tangential relation to many equivalents, and/or there are many other reasons the applicant cannot be expected to describe certain insubstantial substitutes for any claim element amended.

Other embodiments will occur to those skilled in the art and are within the following claims.

What is claimed is:

1. A non-invasive intracranial pressure monitoring system comprising:
a first sensor placed proximate to a perfusion field of an artery of a human subject receiving blood which emanates from the cranial cavity configured to measure pulsations of the artery receiving blood which emanates from the cranial cavity artery and generate first output signals;
a second sensor placed proximate to a perfusion field of an artery of a human subject which does not receive blood emanating from the cranial cavity configured to measure pulsations of the artery which does not receive blood emanating from the cranial cavity and generate second output signals; and
a processing subsystem responsive to the first output signals and the second output signals configured to:
calculate a cross-correlation of the first output signals and the second output signals, and
determine the intracranial pressure of the human subject from a time shift of the cross-correlation with the highest value.

2. The system of claim 1 in which the first sensor and the second sensor include near infrared (NW) sensors.

3. The system of claim 1 in which the first sensor and the second sensor include pressure sensors. The system of claim 1 in which the first sensor and the second sensor include photoplethysmographic sensors.

5. The system of claim 1 in which the first sensor and the second sensor include a combination of one or more of a near infrared (NIR) sensor, a pressure sensor and a photoplethysmographic sensor.

6. The system of claim 1 in which the first sensor is placed proximate one of the supraorbital artery, the supratrocheal artery, or the ophthalmic artery.

7. The system of claim 1 in which the second sensor is placed proximate the external carotid artery or one of its branches.

8. A non-invasive intracranial pressure monitoring system comprising:
a first sensor placed proximate to a perfusion field of an artery of a human subject receiving blond which emanates from the cranial cavity configured to measure pulsations of the artery receiving blood which emanates from the cranial cavity artery and generate first output signals;
a second sensor placed proximate to a perfusion field of an artery of the human subject which does not receive blood emanating from the cranial cavity configured to measure pulsations of the artery which does not receive blood emanating from the cranial cavity and generate second output signals; and
a processing subsystem responsive to the first output signal and the second output signal configured to:
calculate the phase shift of different frequencies included in the first output signals and the second output signals, and
determine intracranial pressure of the human subject from the phase shift at the different frequencies of the first output signals and the second output signals.

9. The system of claim 8 in which the first sensor and the second sensor include near-infrared (NIR) sensors.

10. The system of claim 8 in which the first sensor and the second sensor include pressure sensors.

11. The system of claim 8 in which the first sensor and the second sensor include photoplethysmographic sensors.

12. The system of claim 8 in which the first sensor and the second sensor include a combination of one or more of a near infrared (NIR) sensor, a pressure sensor and a photoplethysmographic sensor.

13. The system of claim 8 in which the first sensor is placed proximate one of the supraorbital artery, the supratrocheal artery, or the ophthalmic artery.

14. The system of claim 9 in which the second sensor is placed proximate the external carotid artery or one of its branches.

15. A non-invasive intracranial pressure monitoring system comprising:
a first sensor placed proximate to a perfusion field of an artery of a human subject receiving Hood which emanates From the cranial cavity configured to measure pulsations of the artery receiving blood which emanates from the cranial cavity artery and generate first output signals;
a second sensor placed proximate to a perfusion field of an artery of the human subject which does not receive blood emanating from the cranial cavity configured to measure pulsations of the artery which does not receive blood emanating from the cranial cavity and generate second output signals; and
a processing subsystem responsive to the first output signal and the second output signal configured to:
calculate a magnitude of different frequencies included in the first output signals and the second output signals, and
determine intracranial pressure of the human subject from a difference in magnitude at the different frequencies of the first output signals and the second output signals.

16. The system of claim 15 in which the first sensor and the second sensor include near-infrared (NIR) sensors.

17. The system of claim 15 in which the first sensor and the second sensor include pressure sensors.

18. The system of claim 15 in which the first sensor and the second sensor include photoplethysmographic sensors.

19. The system of claim 15 in which the first sensor and the second sensor include a combination of one or more of a near infrared (NIR) sensor, a pressure sensor and a photoplethysmographic sensor.

20. The system of claim 15 in which the first sensor is placed proximate one of the supraorbital artery, the supratrocheal artery, or the ophthalmic artery.

21. The system of claim 15 in which the second sensor is placed proximate the external carotid artery or one of its branches.

22. A non-invasive intracranial pressure monitoring system comprising:
a first sensor placed proximate to a perfusion field of an artery of a human subject receiving blood which emanates from the cranial cavity configured to measure pulsations of the artery receiving blood which emanates from the cranial cavity artery and generate first output signals;
a second sensor placed proximate to a perfusion field of an artery of the human subject which does not receive blood emanating from the cranial cavity configured to measure pulsations of the artery which does not receive blood emanating from the cranial cavity and generate second output signals; and
a processing subsystem responsive to the first output signals and the second output signals configured to:
calculate a difference between the first output signals and second output signals, and
determine the intracranial pressure from the difference.

23. The system of claim 22 in which the first sensor and the second sensor include near-infrared sensors.

24. The system of claim 22 in which the first sensor and the second sensor include pressure sensors.

25. The system of claim 22 in which the first sensor and the second sensor include photoplethysmographic sensors.

26. The system of claim 22 in which the first sensor and the second sensor include a combination of one or more of a near infrared (NIR) sensor, a pressure sensor and a photoplethysmographic sensor.

27. The system of claim 22 in which the first sensor is placed proximate one of the supraorbital artery, the supratrocheal artery, or the ophthalmic artery.

28. The system of claim 22 in which the second sensor is placed proximate the external carotid artery or one of its branches.

29. A method for non-invasively determining intracranial pressure, the method comprising:
measuring pulsations of the artery of a human subject receiving blood which emanates from the cranial cavity artery and generating first output signals;
measuring pulsations of the artery of the human subject which does not receive blood emanating from the cranial cavity and generate second output signals;
calculating a cross-correlation of the first output signals and the second output signals; and
determining the intracranial pressure of the human subject from a time shift of the cross-correlation with the highest value.

30. The method of claim 29 in which said measuring pulsations of the artery of a human subject receiving blood which emanates from the cranial cavity artery is performed proximate one of the supraorbital artery, the supratrocheal artery, or the ophthalmic artery.

31. The system of claim 29 in which said measuring pulsations of the artery of the human subject which does not receive blood emanating from the cranial cavity is performed proximate the external carotid artery or one of its branches.

32. A method for non-invasively determining intracranial pressure, the method comprising:
measuring pulsations of the artery of a human subject receiving blood which emanates from the cranial cavity artery and generating first output signals;
measuring pulsations of the artery of the human subject which does not receive blood emanating from the cranial cavity and generate second output signals;
calculating the phase shift of different frequencies included in the first output signals and the second output signals; and
determining intracranial pressure of the human subject from the phase shift at the different frequencies of the first output signals and the second output signals.

33. The method of claim 32 in which said measuring pulsations of the artery of a human subject receiving blood which emanates from the cranial cavity artery is performed proximate one of the supraorbital artery, the supratrocheal artery, or the ophthalmic artery.

34. The system of claim 32 in which said measuring pulsations of the artery of the human subject which does not receive blood emanating from the cranial cavity is performed proximate the external carotid artery or one of its branches.

35. A method for non-invasively determining intracranial pressure, the method comprising:
measuring pulsations of the artery of a human subject receiving blood which emanates from the cranial cavity artery and generating first output signals;
measuring pulsations of the artery of the human subject which does not receive blood emanating from the cranial cavity and generate second output signals;
calculating a magnitude of different frequencies included in the first output signals and the second output signals; and
determining intracranial pressure of the human subject from a difference in magnitude at the different frequencies of the first output signals and the second output signals.

36. The method of claim 35 in which said measuring pulsations of the artery of a human subject receiving blood which emanates from the cranial cavity artery is performed proximate one of the supraorbital artery, the supratrocheal artery, or the ophthalmic artery.

37. The system of claim 35 in which said measuring pulsations of the artery of the human subject which does not receive blood emanating from the cranial cavity is performed proximate the external carotid artery or one of its branches.

38. A method for non-invasively determining intracranial pressure, the method comprising:
measuring pulsations of the artery of a human subject receiving blood which emanates from the cranial cavity artery and generating first output signals;
measuring pulsations of the artery of the human subject which does not receive blood emanating from the cranial cavity and generate second output signals;
calculating a difference between the first output signals and second output signal; and
determining the intracranial pressure from the difference.

39. The method of claim 38 in which said measuring pulsations of the artery of a human subject receiving blood which emanates from the cranial cavity artery is performed proximate one of the supraorbital artery, the supratrocheal artery, or the ophthalmic artery.

40. The system of claim 38 in which said measuring pulsations of the artery of the human subject which does not receive blood emanating from the cranial cavity is performed proximate the external carotid artery or one of its branches.
